(19) United States
(12) Patent Application Publication
(10) Pub. No.: US 2025/0090308 A1
(43) Pub. Date: Mar. 20, 2025

(54) BIORESORBABLE HEART VALVES AND METHODS OF MAKING AND USING SAME

(71) Applicant: Georgia Tech Research Corporation, Atlanta, GA (US)

(72) Inventors: Scott J. Hollister, Atlanta, GA (US); Lakshmi P. Dasi, Atlanta, GA (US); Adam S. Verga, Atlanta, GA (US); Hieu T. Bui, Atlanta, GA (US); Ryan E. Akman, Atlanta, GA (US); Sriharsha Ramaraju, Atlanta, GA (US); Srujana S. Joshi, Atlanta, GA (US); Sanchita S. Bhat, Atlanta, GA (US); Satheesh Kumar Harikrishnan, Atlanta, GA (US)

(21) Appl. No.: 18/729,471

(22) PCT Filed: Jan. 20, 2023
(86) PCT No.: PCT/US2023/061009

§ 371 Date: Jul. 16, 2024

Related U.S. Application Data
(60) Provisional application No. 63/301,673, filed on Jan. 21, 2022.

(51) Int. Cl. A61F 2/24

(52) U.S. Cl. CPC A61F 2/2415; A61F 2/243; A61F 2210/0004; A61F 2210/0014

(57) ABSTRACT

An exemplary embodiment of the present disclosure provides a device for use in cardiovascular interventions, the device comprising a flexible bioresorbable semilunar valve comprising a flexible circumferential body having a ring structure and a plurality of points extending from the ring structure along a longitudinal axis of the valve, a plurality of leaflets extending between the plurality of points, and a flexible cage disposed concentrically around the ring structure, the flexible cage configured to secure the flexible bioresorbable semilunar valve within a cardiac lumen. The device can be 3D printed.

### CROSS-REFERENCE TO RELATED APPLICATIONS

This application claims the benefit of U.S. Provisional Application Ser. No. 63/301,673, filed on 21 Jan. 2022, which is incorporated herein by reference in its entirety as if fully set forth below.

### FIELD OF THE DISCLOSURE

The various embodiments of the present disclosure relate generally to medical devices, and more particularly to heart valves.

### BACKGROUND

Heart valves function to ensure that blood flows in the proper direction within the heart without backwards flow. Valvular heart disease in humans, which compromises the function of the valves and diminish heart functionality, is a common disease that often requires surgical valve repair or replacement. Valvular heart disease can be caused by calcification of the valves with age, and congenital disorders, among other causes. Valvular heart disease is estimated to affect 30 million people and roughly 182,000 heart valve replacements are conducted each year in the U.S. alone. Most of the current solutions involve replacing the native heart valve with either a mechanical valve or a bioprosthetic one, both of which have significant downsides. Significantly, in pediatric applications, pediatric patients can quickly grow out of any permanent implant. There are few adequate options for replacement heart valves in pediatric patients, and the current selection necessitates highly invasive valve replacement surgery. Current manufacturing methods utilized for producing mechanical valves are ill-suited to producing valves that are tailored to individual patient anatomy. Current solutions can also necessitate heavy doses of medication following implantation.

### BRIEF SUMMARY

The present disclosure relates to heart valves, methods of making heart valves, and methods of using heart valves. An exemplary embodiment of the present disclosure provides a device for use in cardiovascular interventions. The device can include a flexible bioresorbable semilunar valve including a flexible circumferential body having a ring structure and a plurality of points extending from the ring structure along a longitudinal axis of the valve and a plurality of leaflets extending between the plurality of points.

In any of the embodiments disclosed herein, the device can further include a flexible cage disposed concentrically around the ring structure, the flexible cage configured to secure the flexible bioresorbable semilunar valve within a cardiac lumen.

In any of the embodiments disclosed herein, the flexible cage can include a patterned surface configured to provide friction with the cardiac lumen and to allow cellular ingrowth.

In any of the embodiments disclosed herein, the patterned surface can be functionalized with one or more of: proteins, peptides, cells, a pharmaceutical agent, biofactors, genetic materials, hydrogels, and biomaterials.

In any of the embodiments disclosed herein, the patterned surface can include a plurality of layers configured to be bioabsorbed over a period of time resulting in a release of the pharmaceutical agent.

In any of the embodiments disclosed herein, the patterned surface can be configured to promote one or more of: cellular ingrowth, tissue adhesion, and vascularization.

In any of the embodiments disclosed herein, the flexible cage can further include an annular member affixed to the flexible cage, the annular member including a first end and an open second end, the first end configured to slide along the annular member past the second end thus gradually transitioning the flexible cage and the annular member from a collapsed delivery configuration to an expanded configuration and to lock the flexible cage in the expanded configuration.

In any of the embodiments disclosed herein, the annular member can include interlocking teeth configured to permit the annular member to transition only from the collapsed delivery configuration to the expanded configuration.

In any of the embodiments disclosed herein, the flexible cage can include a plurality of struts and a plurality of articulating joints, the articulating joints configured to gradually transition the flexible cage from a collapsed delivery configuration wherein the struts are substantially parallel to an expanded configuration wherein the struts are arranged in a diamond pattern and to lock the flexible cage in the expanded configuration.

An exemplary embodiment of the present disclosure provides a system comprising the device and further including a catheter configured to deliver the flexible bioresorbable semilunar valve and the flexible cage.

In any of the embodiments disclosed herein, the flexible cage can include a shape memory material configured to expand at body temperature from collapsed delivery configuration to the expanded configuration.

In any of the embodiments disclosed herein, the system can further include a balloon disposed in the flexible cage and configured to, upon inflating with a fluid, forcibly outwardly expand the flexible cage from the collapsed delivery configuration to the expanded configuration.

In any of the embodiments disclosed herein, a tongue can be formed where each leaflet of the plurality of leaflets adjoins an adjacent leaflet of the plurality of leaflets, and wherein each of the plurality of points of the flexible circumferential body can include a groove configured to receive the tongue.

In any of the embodiments disclosed herein, the device can further include an elastic sleeve configured to be affixed circumferentially around the plurality of leaflets and the flexible circumferential body.

In any of the embodiments disclosed herein, the the flexible circumferential body can include a first material, and the plurality of leaflets can include a second material.

In any of the embodiments disclosed herein, the first material can be stiffer than the second.

In any of the embodiments disclosed herein, the device can further include a reinforcement material connecting the first material and the second material.

In any of the embodiments disclosed herein, the first material can include PCL, and the second material can include APGD.

In any of the embodiments disclosed herein, the the first material can include PCL, and the second material can include MPGD.

In any of the embodiments disclosed herein, the plurality of leaflets can be coupled to the plurality of points with an adhesive.

In any of the embodiments disclosed herein, the plurality of leaflets can be coupled to the plurality of points with an attachment component.

In any of the embodiments disclosed herein, the plurality of leaflets can be coupled to the plurality of points via peg-and-hole connection.

In any of the embodiments disclosed herein, the plurality of leaflets can be coupled to the plurality of points via a suture.

In any of the embodiments disclosed herein, the plurality of leaflets can be fused to the plurality of points via melting.

In any of the embodiments disclosed herein, the plurality of leaflets and the plurality of points can be fused together via a gradient material transition from the plurality of leaflets to the plurality of points.

In any of the embodiments disclosed herein, the sleeve and the plurality of leaflets can form a plurality of lumens where each leaflet of the plurality of leaflets adjoins an adjacent leaflet of the plurality of leaflets, and each of the plurality of points can extend through a respective lumen of the plurality of lumens.

In any of the embodiments disclosed herein, the flexible circumferential body can include a first material, and the plurality of leaflets can include a second material, the first material being stiffer than the second. In any of the embodiments disclosed herein, the first material can include polycaprolactone (PCL), and wherein the second material can include acrylated poly(glycerol dodecanedioate) (APGD) and methacrylated PGD (MPGD).

In any of the embodiments disclosed herein, the plurality of leaflets can be interconnected by a sleeve configured to be affixed over the flexible circumferential body.

In any of the embodiments disclosed herein, the flexible circumferential body can include a first material, and the plurality of leaflets and the sleeve can include a second material, the first material being stiffer than the second.

In any of the embodiments disclosed herein, the flexible cage can include a patterned surface configured to provide friction with the cardiac lumen and to allow cellular ingrowth. In any of the embodiments disclosed herein, the patterned surface can be functionalized with one or more of: proteins, peptides, cells, a pharmaceutical agent, biofactors, genetic materials, and N-ethyl-N′-(3-(dimethylamino)propyl)carbodiimide/N-hydroxysuccinimide (EDC/NHS) chemistry. In any of the embodiments disclosed herein, the patterned surface can include a plurality of layers configured to be bioabsorbed over a period of time resulting in a release of the pharmaceutical agent. In any of the embodiments disclosed herein, the patterned surface can be configured to promote one or more of: cellular ingrowth, tissue adhesion, and vascularization.

In any of the embodiments disclosed herein, the device can further include a catheter configured to deliver the flexible bioresorbable semilunar valve and the flexible cage, and the flexible cage can include a shape memory material configured to expand at body temperature from a collapsed delivery configuration to an expanded configuration.

In any of the embodiments disclosed herein, the flexible circumferential body can further include a patient-specific outer profile formed via additive manufacturing of a three-dimensional valve design determined based on a patient-specific geometry of a cardiac lumen.

In any of the embodiments disclosed herein, the plurality of leaflets can be three-dimensional (3D) printed. In any of the embodiments disclosed herein, the 3D printing can include one or more of the following set: extrusion; selective laser sintering (SLS), direct metal laser sintering (DMLS), digital light processing (DLP), fused deposition modeling (FDM), fused filament fabrication (FFF), PolyJet, stereolithography (SLA), multi jet fusion (MJF), electron beam melting (EBM), solid freeform fabrication (SFF).

In any of the embodiments disclosed herein, the device can further include a pharmaceutical agent embedded throughout the device. In any of the embodiments disclosed herein, the device can be configured to gradually release the pharmaceutical agent.

Another embodiment of the present disclosure provides a device for use in cardiovascular interventions including one or more valve leaflets including one or more bioresorbable materials.

In any of the embodiments disclosed herein, the one or more valve leaflets can be 3D printed. In any of the embodiments disclosed herein, the 3D printing can include one or more of the following set: extrusion; SLS; DMLS; DLP; FDM; FFF; PolyJet; SLA; MJF; EBM; and SFF.

In any of the embodiments disclosed herein, the device can be configured to be delivered transcatheter. In any of the embodiments disclosed herein, the device can have shape memory properties.

In any of the embodiments disclosed herein, the one or more materials can include a biologically resorbable polymer. In any of the embodiments disclosed herein, said biologically resorbable polymer can include any variation, derivative, or combination of one or more of the following: poly(glycerol dodecanedioate) (PGD), APGD, MPGD, poly(ethylene glycol) (PEG), poly(glycerol sebacate) (PGS), polycaprolactone (PCL), poly(diol citrate), and any variations, derivatives, and composites thereof, for example acrylated PGD (APGD), and poly(diol citrate). In any of the embodiments disclosed herein, the one or more materials can include a biologically resorbable metal. In any of the embodiments disclosed herein, the one or more materials can include a biologically resorbable polymer and a biologically resorbable metal. In any of the embodiments disclosed herein, the one or more materials can include a biologically resorbable composite material

In any of the embodiments disclosed herein, the device can further include a leaflet subassembly and a frame configured to support the leaflet subassembly. In any of the embodiments disclosed herein, the frame can include an expandable stent for transcatheter delivery and/or a rigid stent for delivery via an open heart surgery.

In any of the embodiments disclosed herein, the leaflet subassembly can include APGD and MPGD and wherein the frame can include PCL. In any of the embodiments disclosed herein, the device can further include a pharmaceutical active ingredient embedded in or attached to the device. In any of the embodiments disclosed herein, the device can be configured to release the pharmaceutical active ingredient in a user of the device. In any of the embodiments disclosed herein, the device can be configured to provide a delayed release of the pharmaceutical active ingredient in a user of the device.

In any of the embodiments disclosed herein, the device can be tailored to a specific patient's anatomy. Said anatomy can be evaluated using computed tomography CT and/or magnetic resonance imaging (MRI).

In any of the embodiments disclosed herein, the device can further include biofactors. In any of the embodiments disclosed herein, a surface can be functionalized with said biofactors. In any of the embodiments disclosed herein, said biofactors can include one or more selected from the following set: cells; growth factors; genetic material; and pharmaceuticals. In any of the embodiments disclosed herein, said growth factors can include one or more selected from the following set: peptides; proteins; amino acids; and synthetic molecules.

In any of the embodiments disclosed herein, the device can further include one or more selected from the following set: patterned surfaces; and porous scaffolding.

Another embodiment of the present disclosure provides a system for repair of a heart valve. The system can include an apparatus delivered to the heart of a patient through surgical intervention. Said apparatus can include one or more 3D printed components and one or more bioresorbable materials. said apparatus can include: a leaflet subassembly; and a frame subassembly. Said frame subassembly can include one selected from the following set: an expandable stent for transcatheter delivery; and a rigid stent for delivery via open heart surgery.

In any of the embodiments disclosed herein, said surgical intervention can include transcatheter delivery of the apparatus.

In any of the embodiments disclosed herein, said apparatus can have shape memory properties.

In any of the embodiments disclosed herein, said 3D printing can include one or more selected from the following set: extrusion; SLS; DMLS; DLP; FDM; FFF; PolyJet; SLA; MJF; EBM; and SFF.

In any of the embodiments disclosed herein, said bioresorbable materials can include one or more selected from the following set: polymeric material; metallic material; and composite material. Said polymeric material can include any variation, derivative, or combination of one or more selected from the following set: PGD; APGD; MPGD; PCL; PGS; PEG; and poly(diol citrate).

In any of the embodiments disclosed herein, said leaflet subassembly can include APGD, and wherein said frame subassembly can include PCL.

In any of the embodiments disclosed herein, said leaflet subassembly can include MPGD.

In any of the embodiments disclosed herein, a specific anatomy of said patient influences the apparatus. Said anatomy can be evaluated using one or more selected from the following set: CT and MRI.

In any of the embodiments disclosed herein, said apparatus can include biofactors. A surface of the apparatus can be functionalized with said biofactors. Said biofactors can include one or more selected from the following set: cells; growth factors; genetic material; and pharmaceuticals. Said growth factors can include one or more selected from the following set: peptides; proteins; amino acids; and synthetic molecules.

In any of the embodiments disclosed herein, said apparatus can include one or more selected from the following set: patterned surfaces; and porous scaffolding.

In any of the embodiments disclosed herein, said bioresorbable material can be APGD or MPGD, and wherein said 3D printing process can be DLP.

Another embodiment of the present disclosure provides a method of manufacturing a device for use in cardiovascular interventions. The method can include receiving a three-dimensional valve design and additively manufacturing a valve according to the three-dimensional valve design.

In any of the embodiments disclosed herein, the method can further include determining the three-dimensional valve design based on a patient-specific geometry of a cardiac lumen.

In any of the embodiments disclosed herein, determining the three-dimensional valve design can include scanning the cardiac lumen, generating a computer model of the cardiac lumen, and modifying a template valve design based on the computer model with an iterative multiphysics optimization simulation. The iterative multiphysics optimization simulation can be configured to perform one or more of: minimize the valve can include exposing a resin to UV light patterned according to the three-dimensional valve design and thermally curing the valve. The resin can include APGD or MPGD and a photoinitiator.

In any of the embodiments disclosed herein, the method further can include forming the resin, wherein forming the resin can include synthesizing the PGD, and adding the photoinitiator.

In any of the embodiments disclosed herein, additively manufacturing the valve can include selectively solidifying of manufacturing an apparatus used for repair of a heart valve including 3D printing of a bioresorbable material.

In any of the embodiments disclosed herein, said biologically resorbable metal can include one or more of the following: zinc, magnesium, molybdenum, copper and iron, derivatives thereof, alloys thereof, or combinations thereof.

In any of the embodiments disclosed herein, the method can further include designing 505) said apparatus adapted for a patient specific anatomy. In any of the embodiments disclosed herein, said anatomy can be evaluated using one more selected from the following set: CT; and MRI.

In any of the embodiments disclosed herein, the apparatus further can include one or more selected from the following set: a leaflet subassembly; a frame subassembly; a collapsible design; a rigid design; shape memory properties; bio factors incorporated into material bulk; biofactors functionalized to surfaces; patterned surfaces; and porous scaffolding.

In any of the embodiments disclosed herein, said 3D printing can include one or more selected from the following set: extrusion; SLS; DMLS; DLP; FDM; FFF; PolyJet; SLA; MJF; EBM; and SFF. In any of the embodiments disclosed herein, said bioresorbable material can include one or more selected from the following set: polymeric material; metallic material; and composite material. In any of the embodiments disclosed herein, said polymeric material can include any variation, derivative, or combination of one or more selected from the following set: PGD; APGD; MPGD; PCL; PGS; PEG; and poly(diol citrate). In any of the embodiments disclosed herein, said 3D printing of a bioresorbable material can include one or more selected from the following set: 3D printing APGD or MPGD using DLP and 3D printing PCL using SLS.

These and other aspects of the present disclosure are described in the Detailed Description below and the accompanying drawings. Other aspects and features of embodiments will become apparent to those of ordinary skill in the art upon reviewing the following description of specific, exemplary embodiments in concert with the drawings. While features of the present disclosure may be discussed relative to certain embodiments and figures, all embodiments of the present disclosure can include one or more of the features discussed herein. Further, while one or more embodiments may be discussed as having certain advantageous features, one or more of such features may also be used with the various embodiments discussed herein. In similar fashion, while exemplary embodiments may be discussed below as device, system, or method embodiments, it is to be understood that such exemplary embodiments can be implemented in various devices, systems, and methods of the present disclosure.

### BRIEF DESCRIPTION OF THE DRAWINGS

The patent or application file contains at least one drawing executed in color. Copies of this patent or application publication with color drawing(s) will be provided by the Office upon request and payment of the necessary fee.

The following detailed description of specific embodiments of the disclosure will be better understood when read in conjunction with the appended drawings. For the purpose of illustrating the disclosure, specific embodiments are shown in the drawings. It should be understood, however, that the disclosure is not limited to the precise arrangements and instrumentalities of the embodiments shown in the drawings.

FIG. 1 provides an exploded view of a device for use in cardiovascular interventions wherein a plurality of leaflets is interconnected by a sleeve configured to be affixed over a flexible circumferential body, in accordance with an exemplary embodiment of the present disclosure;

FIG. 2 provides an exploded view of a device for use in cardiovascular interventions wherein a plurality of leaflets is interconnected by a sleeve configured to be affixed over a flexible circumferential body, in accordance with an exemplary embodiment of the present disclosure;

FIGS. 3A-3D shows a device in a cardiac lumen, in accordance with an exemplary embodiment of the present disclosure;

FIG. 4A provides a device having a flexible cage, in accordance with an exemplary embodiment of the present disclosure;

FIG. 4B provides a detailed view of the device of FIG. 4A;

FIGS. 5A-5C provide a flexible cage, in accordance with an exemplary embodiment of the present disclosure;

FIG. 6A illustrates a connection between the plurality of leaflets and the plurality of points with an adhesive, in accordance with an exemplary embodiment of the present disclosure;

FIG. 6B illustrates a connection between the plurality of leaflets and the plurality of points with an attachment component, in accordance with an exemplary embodiment of the present disclosure;

FIG. 6C illustrates a connection between the plurality of leaflets and the plurality of points via peg-and-hole connection, in accordance with an exemplary embodiment of the present disclosure;

FIG. 6D illustrates a material gradient connection between the plurality of leaflets and the plurality of points, in accordance with an exemplary embodiment of the present disclosure;

FIG. 6E illustrates a connection between the plurality of leaflets and the plurality of points via a suture, in accordance with an exemplary embodiment of the present disclosure;

FIG. 6F illustrates the plurality of leaflets fused to the plurality of points via melting, in accordance with an exemplary embodiment of the present disclosure;

FIG. 7 illustrates a flexible cage structure with an annular member in accordance with an exemplary embodiment of the present disclosure;

FIGS. 8A-8C illustrate the transition of the annular member from a collapsed state to an expanded state, in accordance with an exemplary embodiment of the present disclosure;

FIGS. 9A-9F provide examples of devices for cardiac intervention, in accordance with an exemplary embodiment of the present disclosure;

FIGS. 10A-10C provide images prototypes of 3D printed heart valves, in accordance with an exemplary embodiment of the present disclosure;

FIGS. 11A-11C provide images of an iteration of a prototype (FIG. 11A) heart valve, in accordance with an exemplary embodiment of the present disclosure. The valve can be seen closing (FIG. 11B) and opening (FIG. 11C) under physiological aortic flow condition via a pulse duplicator;

FIGS. 12A-12C provide an iteration of a prototype (FIG. 12A) heart valve, in accordance with an exemplary embodiment of the present disclosure. The valve can be seen closing (FIG. 12B) and opening (FIG. 12C) under physiological aortic flow condition via a pulse duplicator;

FIGS. 13A-13B illustrate valve leaflets. FIG. 13A shows an isometric of a leaflet with varying thickness. FIG. 13B shows top view showing the same leaflet where an outer portion is 500 um while the inner portion is 200 um thick;

FIGS. 14A-14C illustrates leaflets and portions thereof shaped similar to native heart valve leaflets. FIG. 14A shows leaflets with geometries and thicknesses similar to a healthy adult human. FIG. 14B shows a front and cross-sectional side view of a leaflet. FIG. 14C shows cross-sectional side view of a leaflet, respectively;

FIG. 15A illustrates a cage disposed around a balloon in a collapsed delivery configuration, in accordance with an exemplary embodiment of the present disclosure;

FIG. 15B illustrates a cage disposed around a balloon in an expanded configuration, in accordance with an exemplary embodiment of the present disclosure;

FIG. 16 provides a flow chart depicting a method of manufacturing an APGD embodiment of the device, in accordance with an exemplary embodiment of the present disclosure;

FIG. 17 illustrates Chemical structure of poly(glycerol dodecanedioate), as used in some exemplary embodiments of the present disclosure;

FIG. 18 provides a flow chart of an example design and manufacture process for a device, in accordance with some exemplary embodiments of the present disclosure;

FIGS. 19A-19B provide a flexible semilunar valve, in accordance with an exemplary embodiment of the present disclosure;

FIG. 20A provides a top view of a device during late diastole, in accordance with an exemplary embodiment of the present disclosure;

FIG. 20B provides a top view of a device during late diastole, in accordance with an exemplary embodiment of the present disclosure;

FIG. 20C provides a top view of a device during systole, in accordance with an exemplary embodiment of the present disclosure;

FIG. 20D provides a top view of a device during systole, in accordance with an exemplary embodiment of the present disclosure;

FIG. 20E provides a top view of a device during early diastole, in accordance with an exemplary embodiment of the present disclosure;

FIG. 20F provides a top view of a device during early diastole, in accordance with an exemplary embodiment of the present disclosure;

FIG. 21 provides a top perspective view of a flexible cage, a crimper, and a balloon, in accordance with an exemplary embodiment of the present disclosure;

FIG. 22 provides a plot of Aortic pressure and Ventricular pressure versus time, in accordance with an exemplary embodiment of the present disclosure;

FIG. 23 shows a cross-sectional view of a layered material making up a body of a device, in accordance with an exemplary embodiment of the present disclosure;

FIG. 24 provides a flow chart of an example design and manufacture process for a device, in accordance with an exemplary embodiment of the present disclosure;

FIG. 25 provides a flow chart of an example design and manufacture process for a device, in accordance with an exemplary embodiment of the present disclosure;

FIG. 26 provides a flow chart of an example design and manufacture process for a device, in accordance with an exemplary embodiment of the present disclosure;

FIG. 27 provides a flow chart of an example design and manufacture process for a device, in accordance with an exemplary embodiment of the present disclosure;

FIG. 28 provides a flow chart of an example design and manufacture process for a device, in accordance with an exemplary embodiment of the present disclosure;

FIG. 29 provides a flow chart of an example design and manufacture process for a device, in accordance with an exemplary embodiment of the present disclosure.

### DETAILED DESCRIPTION

To facilitate an understanding of the principles and features of the present disclosure, various illustrative embodiments are explained below. The components, steps, and materials described hereinafter as making up various elements of the embodiments disclosed herein are intended to be illustrative and not restrictive. Many suitable components, steps, and materials that would perform the same or similar functions as the components, steps, and materials described herein are intended to be embraced within the scope of the disclosure. Such other components, steps, and materials not described herein can include, but are not limited to, similar components or steps that are developed after development of the embodiments disclosed herein.

As used herein, the terms “3D printing” and “additive manufacturing” are interchangeable to mean manufacturing processes which rely on the addition of material. These processes can be used in conjunction with traditional manufacturing methods including machining and laser cutting, and the present examples should not be construed as being limited to any particular manufacturing process unless otherwise specified.

As used herein, the terms “biologically resorbable”, “bioresorbable”, and “biodegradable” are interchangeable and interpretable as by persons skilled in the pertinent art. Additionally, a device, only a portion of the device, or a component thereof described herein as bioresorbable can be understood to be fully or only partially bioresorbed.

The present disclosure provides a unique combination of a heart valve that can be both 3D printed and bioresorbable. Some embodiments may further comprise features including patient-specific design, transcatheter delivery, shape memory behavior, collapsible design, functionalization of surfaces, biofactor doped materials, composite materials, patterned surface, and more.

The general utility of 3D printed resorbable heart valves is in heart valve replacement and repair procedures, which can be necessary in the treatment of myriad diseases. Resorbability particularly lends itself to pediatric applications, while printability lends itself to patient-specific applications.

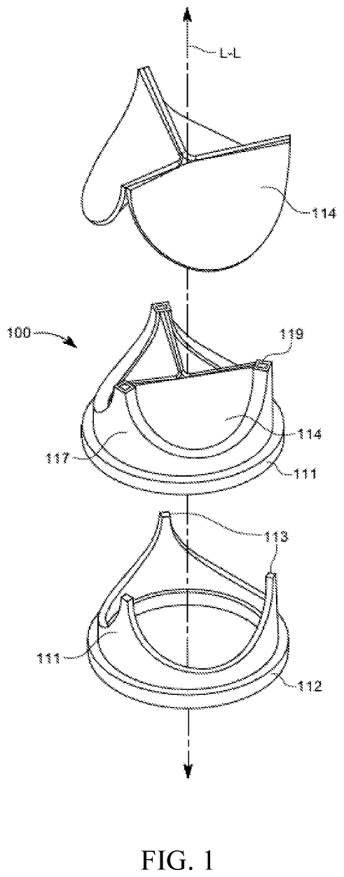

As shown in FIG. 1, an exemplary embodiment of the present disclosure provides a device 100 for use in cardiovascular interventions. The device 100 can include a flexible bioresorbable semilunar valve 110 including a flexible circumferential body 111 having a ring structure 112 and a plurality of points 113 extending from the ring structure 112 along a longitudinal axis L-L of the valve 110 and a plurality of leaflets 114 extending between the plurality of points 113.

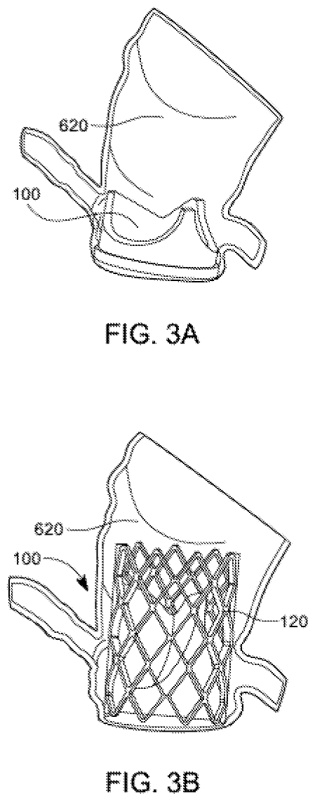

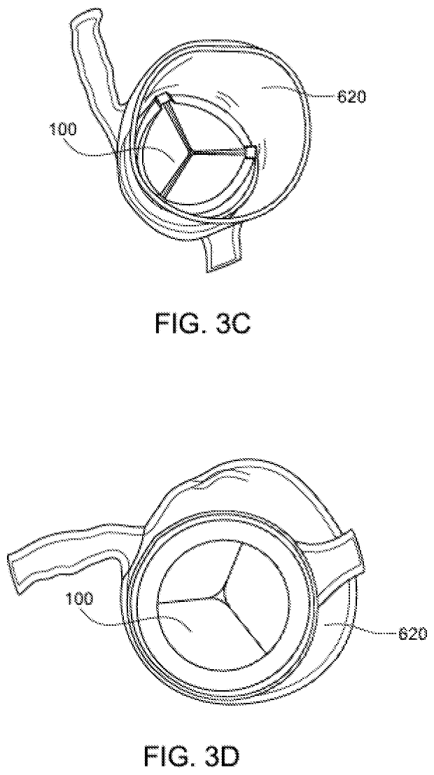

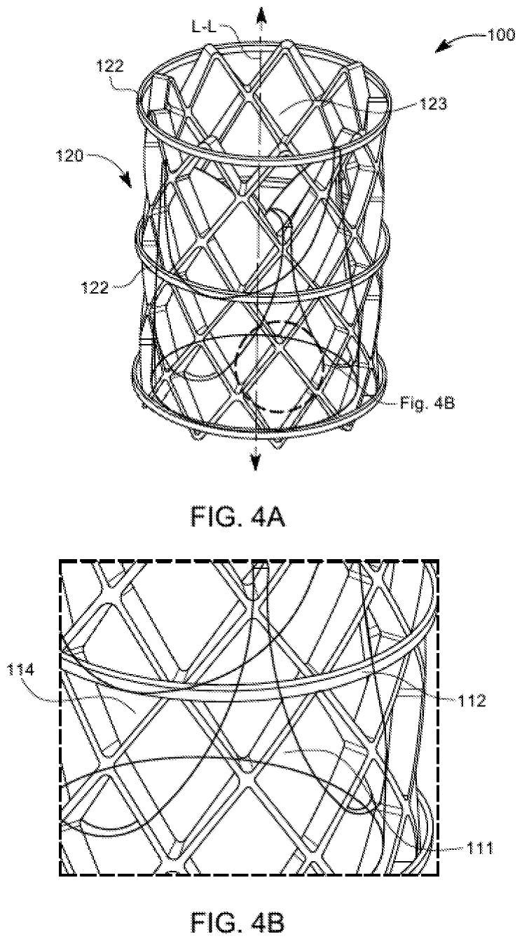

In any of the embodiments disclosed herein, the device 100 can further include a flexible cage 120 (see FIG. 3B, FIG. 4A-4B) disposed concentrically around the ring structure 112. The flexible cage 120 can be configured to secure the flexible bioresorbable semilunar valve 110 within a cardiac lumen 620.

In any of the embodiments disclosed herein, the flexible cage 120 can include a patterned surface 121 configured to provide friction with the cardiac lumen and to allow cellular ingrowth.

In any of the embodiments disclosed herein, the patterned surface 121 can be functionalized with one or more of: proteins, peptides, cells, a pharmaceutical agent, biofactors, genetic materials, hydrogels, and biomaterials.

In any of the embodiments disclosed herein, the patterned surface 121 can include a plurality of layers configured to be bioabsorbed over a period of time resulting in a release of the pharmaceutical agent.

In any of the embodiments disclosed herein, the patterned surface 121 can be configured to promote one or more of: cellular ingrowth, tissue adhesion, and vascularization.

In any of the embodiments disclosed herein, the flexible cage 120 can further include an annular member 125 affixed to the flexible cage 120, the annular member 125 including a first end 125a and an open second end 125b, the first end 125a configured to slide along the annular member 125 past the second end 125b thus gradually transitioning the flexible cage 120 and the annular member 125 from a collapsed delivery configuration 123 to an expanded configuration 124 and to lock the flexible cage 120 in the expanded configuration 124.

In any of the embodiments disclosed herein, the annular member 125 can include interlocking teeth configured to permit the annular member 125 to transition only from the collapsed delivery configuration 123 to the expanded configuration 124.

In any of the embodiments disclosed herein, the flexible cage 120 can include a plurality of struts and a plurality of articulating joints 126, the articulating joints 126 configured to gradually transition the flexible cage 120 from a collapsed delivery configuration 123 wherein the struts are substantially parallel to an expanded configuration 124 wherein the struts are arranged in a diamond pattern and to lock the flexible cage 120 in the expanded configuration 124.

An exemplary embodiment of the present disclosure provides a system comprising the device 100 and further including a catheter configured to deliver the flexible bioresorbable semilunar valve 110 and the flexible cage 120.

In any of the embodiments disclosed herein, the flexible cage 120 can include a shape memory material configured to expand at body temperature from collapsed delivery configuration 123 to the expanded configuration 124.

In any of the embodiments disclosed herein, the system can further include a balloon 140 disposed in the flexible cage 120 and configured to, upon inflating with a fluid, forcibly outwardly expand the flexible cage 120 from the collapsed delivery configuration 123 to the expanded configuration 124.

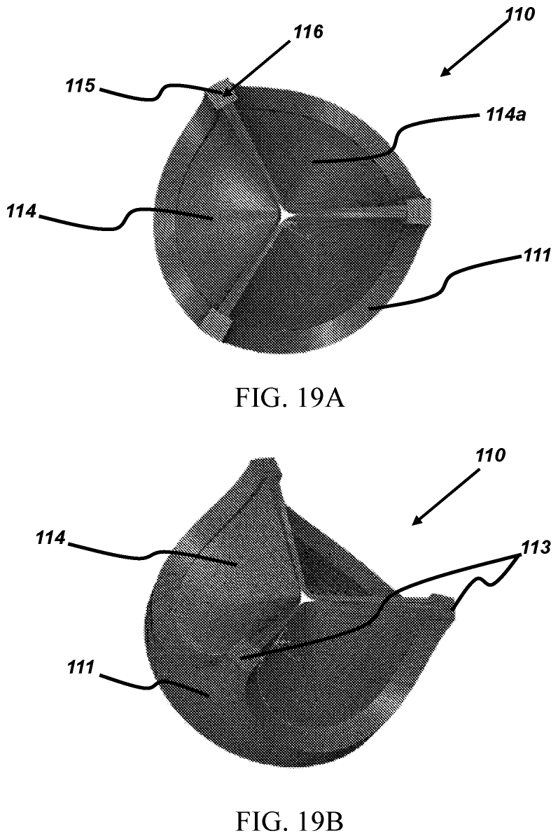

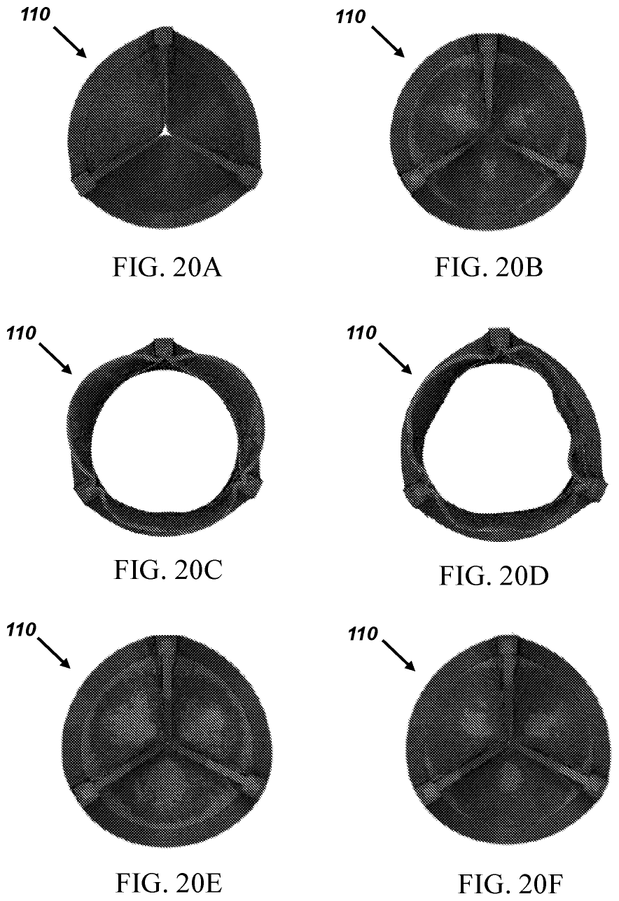

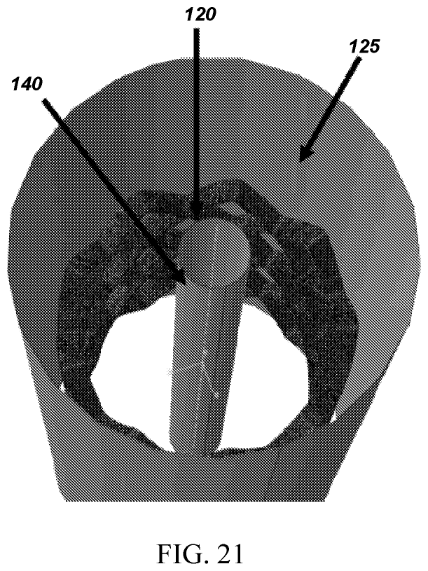

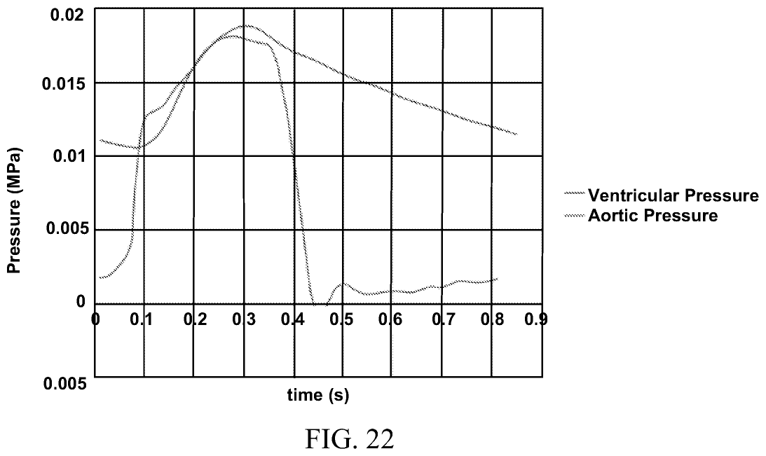

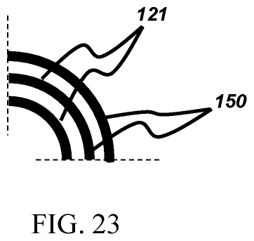

As shown in FIG. 19A, in any of the embodiments disclosed herein, a tongue 115 can be formed where each leaflet of the plurality of leaflets 114 adjoins an adjacent leaflet 114a of the plurality of leaflets 114. As also shown in FIG. 19A, in any of the embodiments disclosed herein, each of the plurality of points 113 of the flexible circumferential body 111 can include a groove 116 configured to receive the tongue 115.

In any of the embodiments disclosed herein, the device 100 can further include an elastic sleeve 117 configured to be affixed circumferentially around the plurality of leaflets 114 and the flexible circumferential body 111.

In any of the embodiments disclosed herein, the the flexible circumferential body 111 can include a first material, and the plurality of leaflets 114 can include a second material.

In any of the embodiments disclosed herein, the first material can be stiffer than the second.

In any of the embodiments disclosed herein, the device 100 can further include a reinforcement material connecting the first material and the second material.

In any of the embodiments disclosed herein, the first material can include PCL, and the second material can include APGD.

In any of the embodiments disclosed herein, the the first material can include PCL, and the second material can include MPGD.

In any of the embodiments disclosed herein, the plurality of leaflets 114 can be coupled to the plurality of points 113 with an adhesive.

In any of the embodiments disclosed herein, the plurality of leaflets 114 can be coupled to the plurality of points 113 with an attachment component.

In any of the embodiments disclosed herein, the plurality of leaflets 114 can be coupled to the plurality of points 113 via peg-and-hole connection.

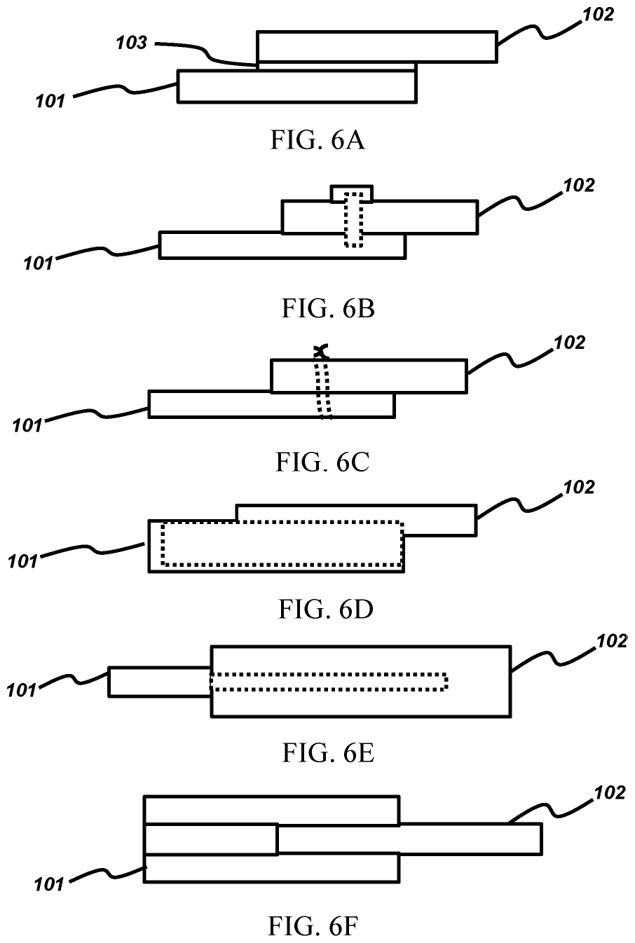

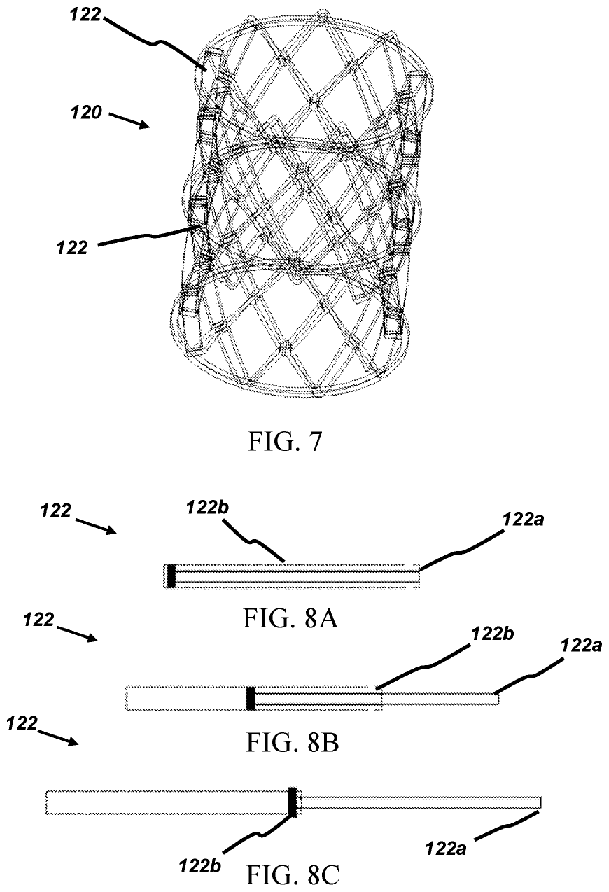

FIGS. 6A-6F show the plurality of leaflets 114 attached to the plurality of points 113.

In any of the embodiments disclosed herein, the plurality of leaflets 114 can be coupled to the plurality of points 113 via a suture.

In any of the embodiments disclosed herein, the plurality of leaflets 114 can be fused to the plurality of points 113 via melting.

In any of the embodiments disclosed herein, the plurality of leaflets 114 and the plurality of points 113 can be fused together via a gradient material transition from the plurality of leaflets 114 to the plurality of points 113.

In any of the embodiments disclosed herein, the sleeve 117 and the plurality of leaflets 114 can form a plurality of lumens where each leaflet of the plurality of leaflets 114 adjoins an adjacent leaflet of the plurality of leaflets 114, and each of the plurality of points 113 can extend through a respective lumen of the plurality of lumens.

In any of the embodiments disclosed herein, the flexible circumferential body 111 can include a first material, and the plurality of leaflets 114 can include a second material. The first material can be stiffer than the second material. In any of the embodiments disclosed herein, the first material can include PCL, and the second material can include APGD or MPGD.

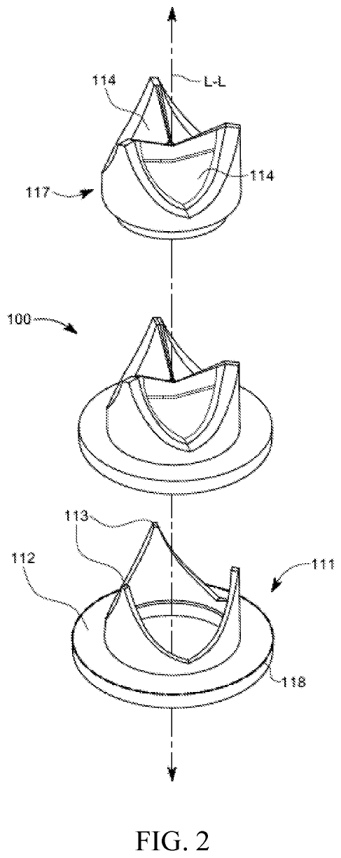

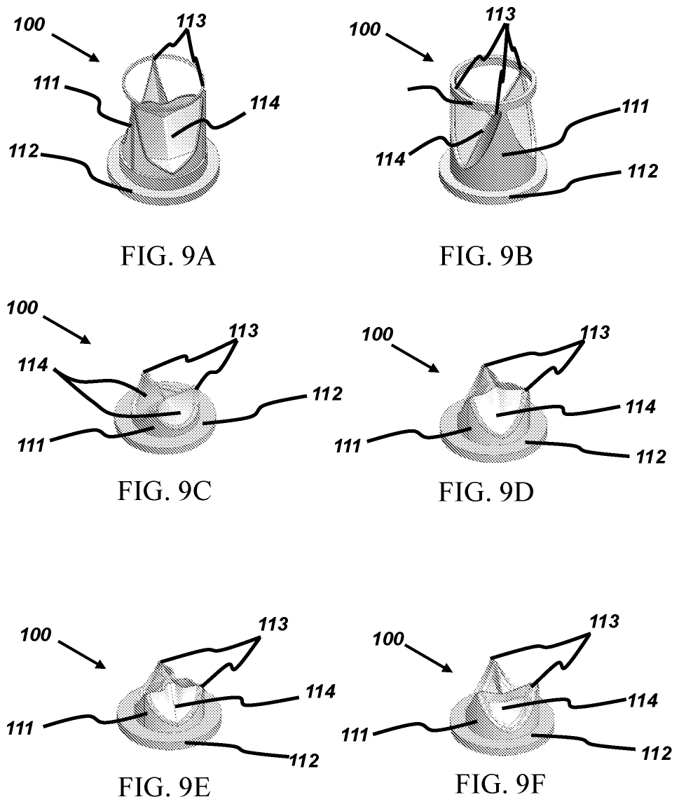

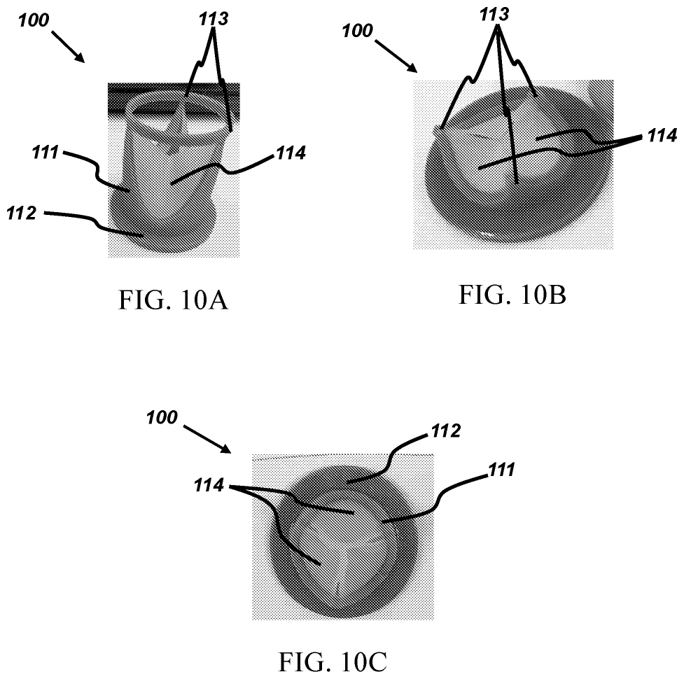

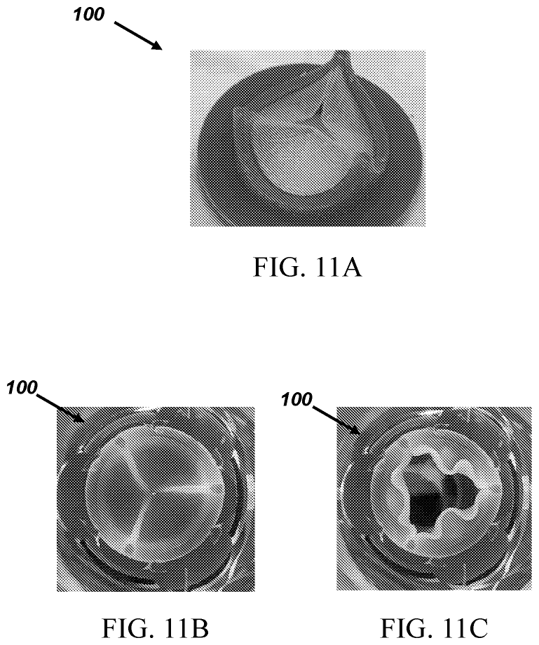

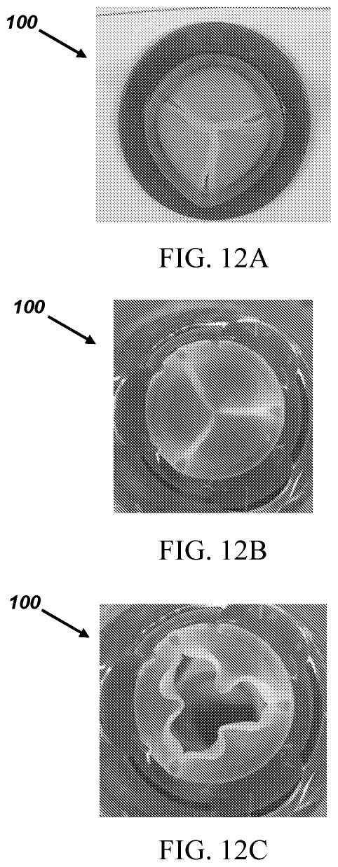

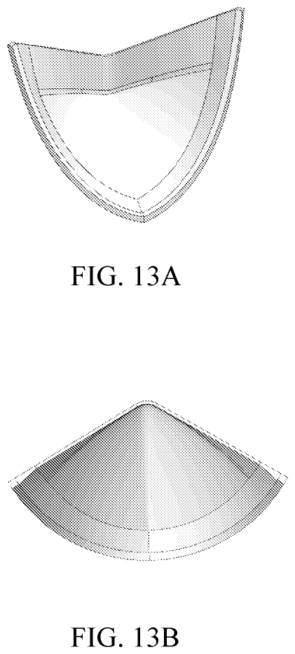

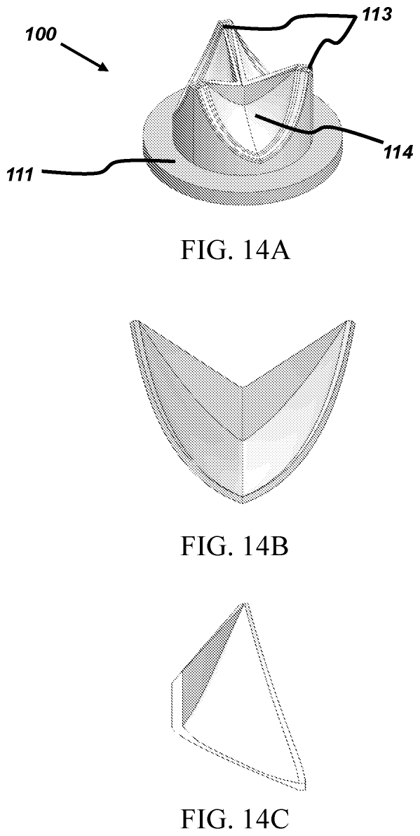

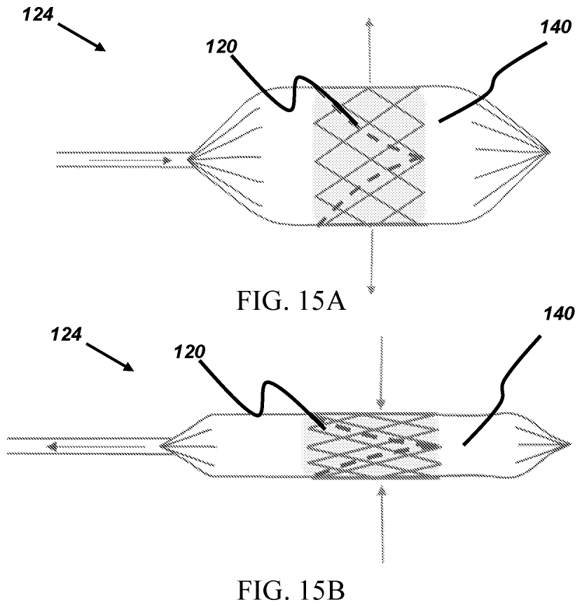

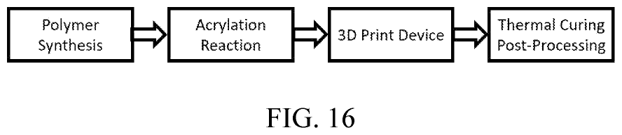

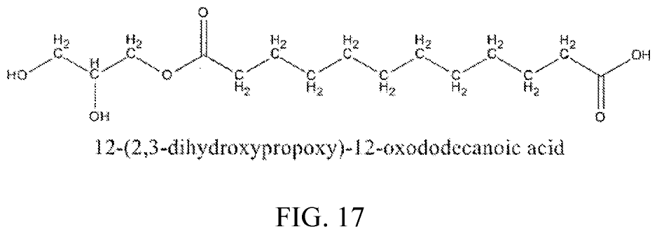

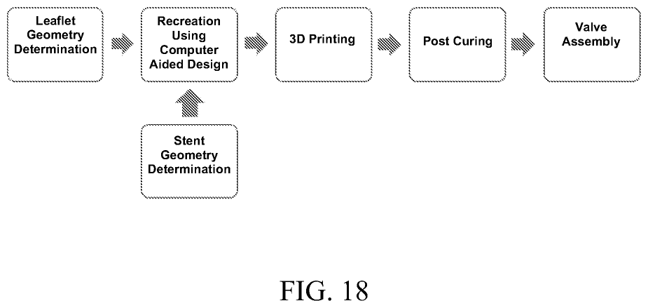

As shown in FIGS. 1-2, FIGS. 9A-9B, in any of the embodiments disclosed herein, the plurality of leaflets 114 can be interconnected by a sleeve 117. The sleeve 117 can be configured to be affixed over the flexible circumferential body 111.

In any of the embodiments disclosed herein, the flexible circumferential body 111 can include a first material, and the plurality of leaflets 114 and the sleeve 117 can include a second material. The first material can be stiffer than the second material.

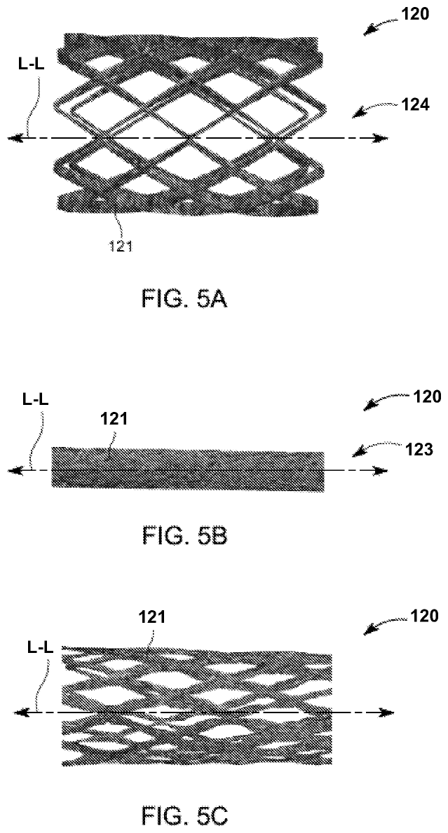

As shown in FIGS. 5A-C, in any of the embodiments disclosed herein, the flexible cage 120 can include a patterned surface 121. The patterned surface 121 can be configured to provide friction with the cardiac lumen 620 and to allow cellular ingrowth. In any of the embodiments disclosed herein, the patterned surface 121 can be functionalized with one or more of: proteins, peptides, cells, a pharmaceutical agent, biofactors, genetic materials, and EDC/NHS chemistry. In any of the embodiments disclosed herein, the patterned surface 121 can include a plurality of layers configured to be bioabsorbed over a period of time resulting in a release of the pharmaceutical agent. In any of the embodiments disclosed herein, the patterned surface 121 can be configured to promote one or more of: cellular ingrowth, tissue adhesion, and vascularization.

In any of the embodiments disclosed herein, the device 100 can further include a catheter configured to deliver the flexible bioresorbable semilunar valve 110 and the flexible cage 120, and the flexible cage 120 can include a shape memory material configured to expand at body temperature from a collapsed delivery configuration 123 to an expanded configuration 124. The flexible cage can be crimped with a crimper 125.

As shown in FIGS. 3A-3D, in any of the embodiments disclosed herein, the flexible circumferential body 111 can further include a patient-specific outer profile 118. The patient-specific outer profile 118 can be formed via additive manufacturing of a three-dimensional valve design determined based on a patient-specific geometry 621 of a cardiac lumen 620. In any of the embodiments disclosed herein, the plurality of leaflets 114 can be 3D printed. In any of the embodiments disclosed herein, the 3D printing can include one or more of the following set: extrusion; SLS; DMLS; DLP; FDM; FFF; PolyJet; SLA; MJF; EBM; and SFF.

In any of the embodiments disclosed herein, the device can further include a pharmaceutical agent embedded throughout the device 100. In any of the embodiments disclosed herein, the device 100 can be configured to gradually release the pharmaceutical agent.

Another embodiment of the present disclosure provides a device 100 for use in cardiovascular interventions including one or more valve leaflets 114 including one or more bioresorbable material(s) 150.

In any of the embodiments disclosed herein, the one or more valve leaflets 114 can be 3D printed. In any of the embodiments disclosed herein, the 3D printing can include one or more of the following set: extrusion; SLS; DMLS; DLP; FDM; FFF; PolyJet; SLA; MJF; EBM; and SFF.

In any of the embodiments disclosed herein, the device 100 can be configured to be delivered transcatheter.

In any of the embodiments disclosed herein, the device 100 can have shape memory properties.

In any of the embodiments disclosed herein, the one or more materials can include a biologically resorbable polymer. In any of the embodiments disclosed herein, said biologically resorbable polymer can include any variation, derivative, or combination of one or more of the following: PGD; APGD; MPGD; PCL; PGS; PEG; and polydiol citrate. In any of the embodiments disclosed herein, the one or more materials can include a biologically resorbable metal. In any of the embodiments disclosed herein, the one or more materials can include a biologically resorbable polymer and a biologically resorbable metal. In any of the embodiments disclosed herein, the one or more materials can include a biologically resorbable composite material

In any of the embodiments disclosed herein, the device can further include a leaflet subassembly 114 and a frame 112 configured to support the leaflet subassembly 114. In any of the embodiments disclosed herein, the frame 112 can include an expandable stent 120 for transcatheter delivery and/or a rigid stent for delivery via an open heart surgery.

In any of the embodiments disclosed herein, the leaflet subassembly 114 can include APGD or MPGD and wherein the frame 112 can include PCL. In any of the embodiments disclosed herein, the device can further include a pharmaceutical active ingredient embedded in or attached to the device 100. In any of the embodiments disclosed herein, the device 100 can be configured to release the pharmaceutical active ingredient in a user of the device 100.

In any of the embodiments disclosed herein, the device 100 can be configured to provide a delayed release of the pharmaceutical active ingredient in a user of the device 100.

In any of the embodiments disclosed herein, the device 100 can be tailored to a specific patient's anatomy 600. Said anatomy can be evaluated using CT and/or MRI.

In any of the embodiments disclosed herein, the device can further include biofactors.

In any of the embodiments disclosed herein, a surface can be functionalized with said biofactors. In any of the embodiments disclosed herein, said biofactors can include one or more selected from the following set: cells; growth factors; genetic material; and pharmaceuticals.

In any of the embodiments disclosed herein, said growth factors can include one or more selected from the following set: peptides; proteins; amino acids; and synthetic molecules.

In any of the embodiments disclosed herein, the device can further include one or more selected from the following set: patterned surfaces 121; and porous scaffolding.

Another embodiment of the present disclosure provides a system 300 for repair of a heart valve. The system 300 can include an apparatus 200 delivered to the heart of a patient 600 through surgical intervention. Said apparatus 200 can include one or more 3D printed components and one or more bioresorbable materials. Said apparatus 200 can include: a leaflet subassembly 114; and a frame subassembly 112. Said frame subassembly 112 can include one selected from the following set: an expandable stent for transcatheter delivery; and a rigid stent for delivery via open heart surgery.

In any of the embodiments disclosed herein, said surgical intervention can include transcatheter delivery of the apparatus 200.

In any of the embodiments disclosed herein, said apparatus 200 can have shape memory properties.

In any of the embodiments disclosed herein, said 3D printing can include one or more selected from the following set: extrusion; SLS; DMLS; DLP; FDM; FFF; PolyJet; SLA; MJF; EBM; and SFF.

In any of the embodiments disclosed herein, said bioresorbable materials can include one or more selected from the following set: polymeric material; metallic material; and composite material. Said polymeric material can include any variation, derivative, or combination of one or more selected from the following set: PGD; APGD; MPGD; PCL; PGS; PEG; and polydiol citrate.

In any of the embodiments disclosed herein, said leaflet subassembly 114 can include APGD or MPGD, and wherein said frame subassembly 112 can include PCL.

In any of the embodiments disclosed herein, a specific anatomy of said patient 600 influences the apparatus 200. Said anatomy can be evaluated using one or more selected from the following set: CT and MRI.

In any of the embodiments disclosed herein, said apparatus 200 can include biofactors. A surface of the apparatus 200 can be functionalized with said biofactors. Said biofactors can include one or more selected from the following set: cells; growth factors; genetic material; and pharmaceuticals. Said growth factors can include one or more selected from the following set: peptides; proteins; amino acids; and synthetic molecules.

In any of the embodiments disclosed herein, said apparatus 200 can include one or more selected from the following set: patterned surfaces; and porous scaffolding.

In any of the embodiments disclosed herein, said bioresorbable material can be APGD or MPGD, and wherein said 3D printing process can be DLP.

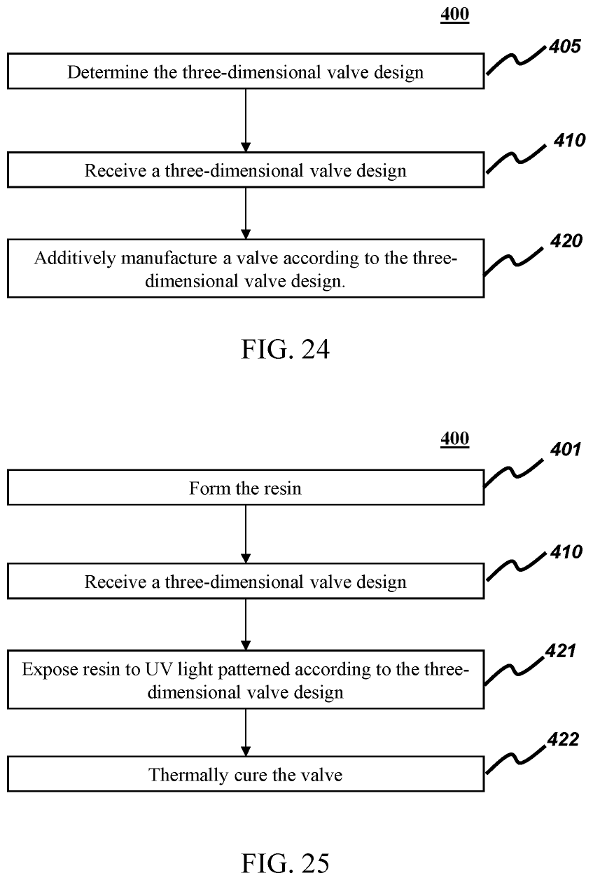

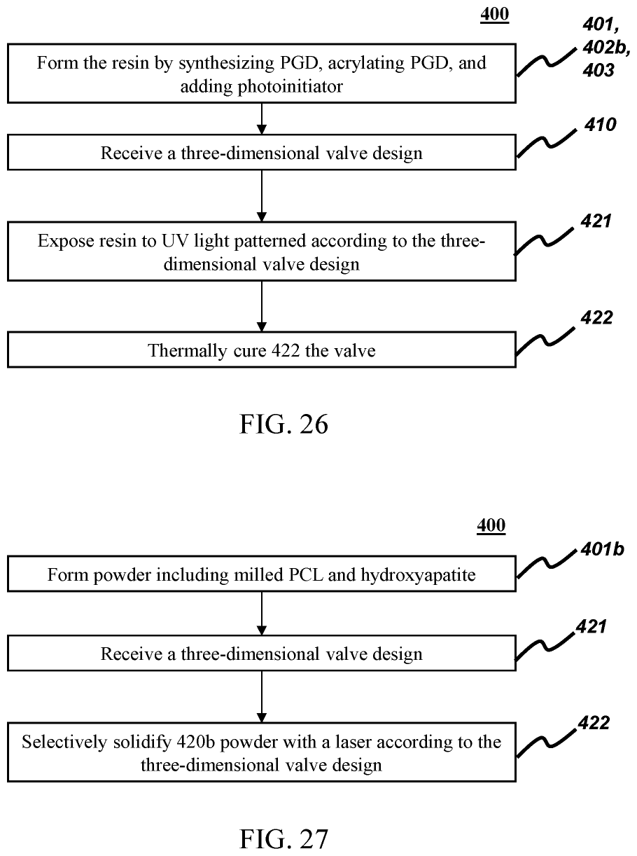

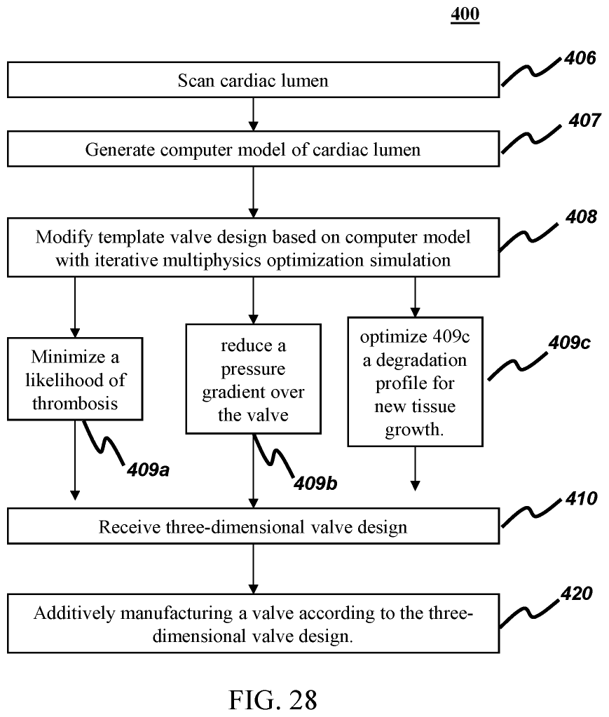

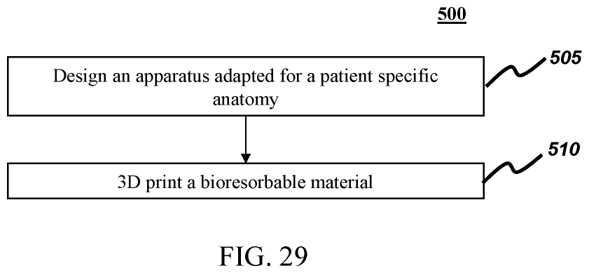

FIGS. 24-29 show methods of manufacturing a device. Another embodiment of the present disclosure provides a method 400 of manufacturing a device for use in cardiovascular interventions. The method 400 can include receiving 410 a three-dimensional valve design and additively manufacturing 420 a valve according to the three-dimensional valve design.

In any of the embodiments disclosed herein, the method 400 can further include determining 405 the three-dimensional valve design based on a patient-specific geometry of a cardiac lumen.

In any of the embodiments disclosed herein, determining 405 the three-dimensional valve design can include scanning 406 the cardiac lumen, generating 407 a computer model of the cardiac lumen, and modifying 408 a template valve design based on the computer model with an iterative multiphysics optimization simulation. The iterative multiphysics optimization simulation can be configured to perform one or more of: minimize 409a a likelihood of thrombosis, reduce 409b a pressure gradient over the valve, or optimize 409c a degradation profile for new tissue growth.

In any of the embodiments disclosed herein, additively manufacturing 420 the valve can include exposing 421 a resin to UV light patterned according to the three-dimensional valve design and thermally curing 422 the valve. The resin can include APGD or MPGD and a photoinitiator.

In any of the embodiments disclosed herein, the method 400 further can include forming 401 the resin, wherein forming the resin can include synthesizing 402b PGD, acrylating 403 the PGD, and adding 404 the photoinitiator.

In any of the embodiments disclosed herein, additively manufacturing 420 the valve can include selectively solidifying 420b a powder with a laser according to the three-dimensional valve design. The powder can include milled PCL and hydroxyapatite and the method 400 can include forming 401b the powder.

Another embodiment of the present disclosure provides a method 500 of manufacturing an apparatus used for repair of a heart valve including 3D printing 510 of a bioresorbable material.

In any of the embodiments disclosed herein, said biologically resorbable metal can include one or more of the following: zinc, magnesium, molybdenum, copper and iron, derivatives thereof, alloys thereof, or combinations thereof.

In any of the embodiments disclosed herein, the method 500 can further include designing 505 said apparatus adapted for a patient specific anatomy. In any of the embodiments disclosed herein, said anatomy can be evaluated using one more selected from the following set: CT; and MRI.

In any of the embodiments disclosed herein, the apparatus further can include one or more selected from the following set: a leaflet subassembly; a frame subassembly; a collapsible design; a rigid design; shape memory properties; biofactors incorporated into material bulk; biofactors functionalized to surfaces; patterned surfaces; and porous scaffolding.

In any of the embodiments disclosed herein, said 3D printing can include one or more selected from the following set: extrusion; SLS; DMLS; DLP; FDM; FFF; PolyJet; SLA; MJF; EBM; and SFF. In any of the embodiments disclosed herein, said bioresorbable material can include one or more selected from the following set: polymeric material; metallic material; and composite material. In any of the embodiments disclosed herein, said polymeric material can include any variation, derivative, or combination of one or more selected from the following set: PGD; APGD; MPGD; PCL; PGS; PEG; and polydiol citrate. In any of the embodiments disclosed herein, said 3D printing of a bioresorbable material can include one or more selected from the following set: 3D printing APGD or MPGD using DLP and 3D printing PCL using SLS.

The following examples further illustrate aspects of the present disclosure. However, they are in no way a limitation of the teachings or disclosure of the present disclosure as set forth herein.

### Examples

An exemplary device comprises a bioresorbable material that has been 3D printed into a geometry which allows it to function as a heart valve within a patient. Some embodiments comprise two or more subassemblies, for example leaflets and a frame, while other embodiments comprise a single assembly. Such assemblies may comprise any number of components, and some embodiments of the device comprise only a single component. A frame may be utilized to maintain structural support and affix the device to its intended location within the body. Note that the terms bioresorbable and biodegradable may be used interchangeably.

The 3D printing of the device could be conducted using any number of 3D printing methods. Some components may be manufactured using a different method and then assembled with the 3D printed component(s). Choice of materials and geometry guide the 3D printing method used. Some embodiments of the manufacturing process include but are not limited to extrusion, selective laser sintering (SLS), direct metal laser sintering (DMLS), digital light processing (DLP), fused deposition modeling (FDM), fused filament fabrication (FFF), PolyJet, stereolithography (SLA), multi jet fusion (MJF), electron beam melting (EBM), solid freeform fabrication (SFF), any combination thereof, and any other additive manufacturing technique existing or to be developed in the future. The material, prior to printing, could be in any form, including but not limited to resin, melt, filament, and powder. There may be processing before and/or after printing. In some embodiments this processing comprises thermally curing a resin after printing to improve mechanical strength.

The device could be composed of any number of materials. Polymeric materials could be utilized for none, some but not all, or all components, which may include but are not limited to materials such as poly(glycerol dodecanedioate) (PGD), poly(ethylene glycol) (PEG), poly(glycerol sebacate) (PGS), polycaprolactone (PCL), poly(diol citrate), and any variations, derivatives, and composites thereof, for example acrylated PGD (APGD) and methacrylated PGD (MPGD). Some embodiments may comprise composite materials in order to match the mechanical behavior of specific tissues, for example by layering an organic matrix into a polymer. While polymers are the traditional materials for applications such as this, there are also metallic materials which can be 3D printed and are bioresorbable. Metallic materials could be utilized for none, some but not all, or all components, which may include but are not limited to materials such as iron, zinc and magnesium-based alloys. Some embodiments comprise a frame subassembly composed of metallic material(s) and leaflets subassembly composed of polymeric material(s).

Components may comprise more than one material. For example, materials may be layered, coated, or combined in another fashion in order to optimize the properties of the component. One example of the leaflets includes extracellular matrix, for example in the form of small intestinal submucosa (SIS), embedded into APGD leaflets. This SIS may be in sheets and may be of one or multiple layers, or may be in the form of particulates, which may distributed uniformly or non-uniformly throughout the APGD leaflets.

Degradable materials are utilized to accommodate annulus and leaflet growth, but may also be applied to adult patients. Degradable materials used in some embodiments will resorb in an approximately 3 month to 4 year time frame, depending on materials. Material changes lead to changes in both elastic and post-yield mechanical behavior. For example, Polycaprolactone becomes stiffer during degradation (due to increased crystallinity, but simultaneously more brittle with reduction of post-yield behavior. Furthermore, degradable valves require tissue ingrowth/ongrowth concurrent with material degradation to maintain function.

Biodegradable metal alloys that can be used maybe manufactured by using zinc, magnesium, molybdenum, copper and iron. These alloys have been investigated to reveal excellent biodegradable characteristics and have significant advantages in a load bearing application, such as a heart valve replacement

Non-biodegradable metal alloys maybe used to manufacture certain elements of the stent frame such as the commissures and the clicking components, to avoid premature failure of the device. These maybe medical grade metal alloys such as those widely used, namely cobalt-chromium, stainless steel and nitinol.

Non-metallic materials such as biodegradable polymers including, but not limited to, polycaprolactone (PCL), polyglycolide (PGA), poly(lactic-co-glycolic acid (PLGA) among other similar materials can be used to duplicate the superelastic properties of metals in certain or all embodiments of the stent frame.

Properties that are imperative for this application is good tensile strength (greater than 100 MPa, which is the tensile strength for current FDA approved devices) and high durability that can be achieved by optimal material attachment and design, among other properties.

Embodiments of devices disclosed herein can be constructed according to the materials and material properties in the following tables:

TABLE 1

| | Mass | Hyperelastic (Neo Hooke) | | |
|---|---|---|---|---|
| Material | Density | C10 | D1 | Component(s) |
| Agilus Black | 1e−09 | 0.1084 | 0.09225 | Leaflets, Body |
| Vero Clear | 1e−09 | 306.65 | 3.261e−05 | Stent Post |

TABLE 2

| | Mass Density | Young's | Poisson's | |
|---|---|---|---|---|
| Material | (Mg/mm³) | Modulus | Ratio | Components |
| PCL | 1.14e−09 | 200 | 0.3 | Stent Post |

TABLE 3

| | Mass | Hyperelastic (Neo Hooke) | | |
|---|---|---|---|---|
| Material | Density | C10 | D1 | Component(s) |
| PGD | 1.131e−09 | 0.21 | 0.04762 | Leaflets, Body |

Patterned surfaces are utilized in some embodiments to achieve an optimal interface with surrounding tissues. Patterning may be used for any number of purposes, which may include but are not limited to providing the friction necessary to secure the device in place and encouraging cell growth and/or adhesion. Some embodiments are functionalized, for example with proteins, peptides, and/or other molecules, which may be used for example to promote one or more of the behaviors including cell growth, tissue adhesion, proliferation, and vascularization. To encourage tissue growth of degradable leaflets, polymer surfaces may be functionalized with peptides. These peptides can enhance host cell attachment to leaflet materials and stents to ensure adaptation of implants valves to blood vessel growth. Functionalization can be conducted using N-ethyl-N′-(3-(dimethylamino)propyl)carbodiimide/N-hydroxysuccinimide (EDC/NHS) chemistry in some of these embodiments. The porosity of any given component may vary between embodiments, with some utilizing and open scaffolding, others a denser lattice, and others with low porosity. The resorbable valve can act as a scaffold and improve water intake to promote valve remodeling in some embodiments. The surface could be modified in any number of other ways to achieve, for example, optimal hydrophobicity or pH. Some embodiments also perform biofactor delivery, which may include but is not limited to cells, proteins, genes, peptides, and drugs. For example, some embodiments may have their surface and/or bulk doped with any number of molecules for any number of purposes, including but limited to the delivery of drugs, and which may or may not be released, which may occur over any timeline. Another example of biofactor delivery is the delivery of cells, possibly cells harvested from the patient, which are incorporated into the device, perhaps via cell printing or ingrowth during an in vitro incubation period, or via any other method.

The surgical delivery method may play a large role in the design. Embodiments which are delivered transcatheter can be designed to expand, while embodiments delivered via open heart surgery may not have this feature. Such expansion when being deployed from a catheter could utilize any number of possible methods. As an example only, this may be achieved using shape memory polymers pre-programmed to expand at body temperature, or one or more hinges with tension applied to open them once able, or any other metallic or polymeric material and structural design combination which results in the ability to collapse to the inner diameter of a catheter and proceed to be able to expand, on its own or with assistance, to an optimal diameter with stresses that will keep it well secured in its proper location. The surgical valve replacement can be sutured to the ring structure using traditional suture techniques, except fewer sutures can be used as compared to 12-15 in traditional surgeries. The valve can be aligned to the native commissures and sutured in place through the suture cuff, which will be implemented with a cuff suitable for surgical applications.

A surgical heart valve (SHV) can consist of a polymeric strut that accommodates the strut provision for suture ring as well. The strut has three points that can act as the inner frame on which the leaflet assembly, attached to the outer elastic surface, slides in.

The leaflets printed along with the outer elastic surface consist of the skirt region between each commissure of the leaflet that extends up to the annulus of the valve in the suture ring.

The elastic surface slide on the solid inner strut can be attached to the to prevent the elastic outer frame from moving and deforming during the valve function.

Many current stents are delivered transcatheter, and methods utilized for that application could have utility in this application as well.

Transcatheter heart valve replacement (THV) is an alternative lower risk procedure approved currently for patients who are moderate to high risk for surgery, as compared to traditional gold standard surgical heart valve replacement (SHV). THV's are often deployed by crimping the replacement valve over a deflated balloon, which is placed above a guide wire. The entire assembly is put into a delivery system, which is often then routed to the aorta through the femoral artery in the groin. In some cases, for example those where the patient has calcification or pathophysiological elements in the femoral artery, the deployment can be done via the carotid artery or directly via apical incision. Since THV presents a unique environment for the device during deployment, the stent members need to be constructed out of super elastic/high strength medical grade materials. There are materials—both metal and polymeric, currently used in the market for balloon expandable procedures. Although the currently approved metallic alloys are medical grade and widely successful, they do not come without drawbacks. More specifically, the device experiences immune rejection, endocarditis, thrombosis and hypo-attenuated leaflet thickening due to the use of non-biodegradable stents and animal tissue as leaflets. Although this tissue is fixed and treated, it does not have the capacity to degrade in the body therefore prompting an immune rejection. Eventually, in case of extreme adverse effects, a redo-THV is placed inside the existing prosthesis if the patient remains moderate to high risk for surgery, further complicating the fluid mechanics and prompting flow stasis in those regions.

In one example, a transcatheter heart valve can include a metallic or polymeric stent with three components on the longitudinal frame of the stent that will have elements with expandable properties. Namely, these elements will have the capacity to radially expand and click in place, when the desired radius/shape is achieved.

The THV can include three-dimensional leaflets along with a skirt region, to prevent paravalvular leakage. The skirt region will be from the inlet of the valve extending to the frame and ending where the edge of the leaflets will begin.

Expandable elements can be included to control the radial expansion as needed, and to also allow for self-expanding elements within a balloon expanded deployment. The balloon can serve as a guide to situate the THV inside the anatomy after which the balloon's radial force will enable a locking annular member to maintain the radial expansion.

The transcatheter delivery mechanism can include a crimper that will crimp the entire valve assembly to a size that is compatible to the balloon and catheter size to be used for the specific case. The valve can be crimped over a balloon and a guidewire catheter, which will be used to deploy the valve and guide the placement of the valve respectively. After the entire balloon, wire, and valve assembly is inserted through the patient's access point (for example, femoral, apical, carotid), the other end of the balloon can be equipped with a lock mechanism, which will be used to insert saline to expand the balloon after which it will be closed off to air.

In some examples, the leaflets can be manufactured via additive manufacturing techniques and the stent members can be manufactured via additive manufacturing, machining, casting to achieve the required shape. This example can include a skirt region beneath the free edge of the leaflet and above the inlet of the stent to prevent paravalvular leakage. The skirt will also help the device to anchor to the diseased (mostly calcified) annulus, therefore preventing downstream embolization of the device. The valve assembly will either be manufactured as a complete entity using polymeric materials and combinations of polymers, or separate stent and leaflet entities will be manufactured and assembled using sutures or other adhering techniques. When completely expanded, the stent will be between 10 mm-30 mm (depending on an intra annular or supra-annular design). The stent frame may have several configurations of various design elements comprising of the total frame including number of bars, intersections and struts and their respective thicknesses. These will depend on a variety of things such as deployment height, radial force that will be exerted by the balloon and the capability of the design to sustain it, and shape of the stent profile. The stent will maintain an optimal valve orifice area and provide structural support to the device assembly, avoiding catastrophic or premature failure of the device. The stent when expanded, will push against the native anatomy and pathophysiological structures (calcium). Embolization of calcium fragments can cause strokes and must be avoided. The frame when fully expanded experiences pressure from the balloon that is high enough to expand the stent and deploy it, although not large enough to distend or damage the native aorta. The whole valve assembly, when crimped, will be between 4-6 mm, which is the general diameter of the femoral artery, to ensure smooth delivery and avoid endothelial damage. The frame of the stent when crimped will be between approximately 20 mm-40 mm (depending on an intra annular or supra-annular design), to completely encapsulate the balloon and ensure that it lies in the center of the balloon, avoiding asymmetric expansion.

The leaflets can be assembled over this manufactured stent frame using sutures at commissures and along the inlet curvature of the valve frame to prevent leakage.

In some examples of this device the leaflets and stent frame can be separate pieces combined to make the device. In these examples the leaflets can be adhered to the stent frame in one of multiple ways. Adhering methods include but are not limited to the use of sutures, glue adhesives, physical peg-hole connection or press fitting, melt adhesion, and a gradient material approach from the frame to the leaflets. Sutures can be wrapped around the stent frame and leaflets or can penetrate both the leaflet and frame material to connect the two parts. Glue adhesives, including but not limited to, like cyanoacrylates, surgical glues, or bioinspired photocurable adhesives can be used to connect the stent frame to the leaflets. The physical design of the stent frame and leaflets can also lend themselves to their adhesion. The stent frame and leaflets can be designed with complementary pegs and holes allowing for a peg-hole connection to adhere the two parts together. The stent frame can also be designed in such a way that the leaflets can snugly fit into the frame itself directly allowing for adhesion via press fitting. Another example, depending on the material used for the stent frame, is the controlled melting of connective elements on the frame directly onto the leaflets, which once cooled would provide adhesion between the two parts of the device. Another possible method of adhesion is manufacturing the two parts together via a method such as 3D printing, where the frame material can be extruded continuously with the leaflet material. In this instance a gradient can be formed with material for the frame forming a gradient with material in the leaflets starting from the frame and continuing into the leaflets thus connecting the two parts and making them into a single device.

In some examples, the stent frame can have a non-circular inlet to reflect the native annulus of the patient. For a patient-specific approach, echocardiography can be used to map the patient's annulus through the cardiac cycle, along with other parameters such as sinus diameter, leaflet and sinus heights, to create the non-circular inlet. The valve frame may also be modified to account for no coronary artery construction post-THV, by evaluating the risk of construction through calculation of coronary height. A dilated ventricle (caused by disease) may remodel the aorta to have a dilated aortic annulus (particularly in patients presenting with aortic insufficiency). The THV frame can be enlarged to fit this dilated annulus, by making use of the clicking mechanism on the farthest setting.

The percutaneous delivery method may play a large role in the design. Embodiments which are delivered transcatheter would need to be designed to expand, while embodiments delivered via open heart surgery would not have this requirement. Such expansion when being deployed from a catheter could utilize any number of possible methods. For illustration only, this may be achieved using shape memory polymers pre-programmed to expand at body temperature, or one or more hinges with tension applied to open them once able, or any other metallic or polymeric material and structural design combination which results in the ability to collapse to the inner diameter of a catheter and proceed to be able to expand, on its own or with assistance, to an optimal diameter with stresses that will keep it well secured in its proper location. Many current stents are delivered transcatheter, and methods utilized for that application could have utility in this application as well.

In some examples, the frame of a transcatheter valve embodiment may be fabricated through 3D printing, additive manufacturing, or rapid prototyping. Material composition could comprise polymers, metals, alloys, composites, or any other material or combination thereof. In such embodiments, the frame can be a stent or of any other design allowing for the crimping and expansion of the valve; it can be self-expandable or manually expandable through techniques such as balloon dilation. Some embodiments of the frame can fold up for transcatheter delivery and then unfold and lock into an open/expanded position once deployed to their intended location. This unfolding process may be facilitated by tension springs, a balloon (for example balloon 140), shape memory properties, or by any other mechanism. Once unfolded, the frame locks into position, which may be facilitated by any number of mechanisms, for example internal stresses, friction between components, and hole-hook, snap-fit tabs, or other similar fastening techniques.

To prepare for 3D printing, the valve, and its components, which may include but are not limited to a frame and leaflets, may be created using any computer-aided design (CAD) software, such as SolidWorks and AutoCAD. Parameters to account for may include but are not limited to leaflet height, fixed edge's dimensions, the free edge's geometry, and the diameter of the valve. These parameters can be acquired from the geometry of naturally derived leaflets, which can be either an idealized version or patient specific. The latter may be important for custom designing heart valves to a specific patient's need, such as mitigating the patient's particular medical problem, including valve stenosis, aortic bicuspid valve, valve insufficiency, and congenital heart defects.

To incorporate physiological leaflet elements, anisotropy can be imparted on various components of the device. Leaflet anisotropy is a native phenomenon, which is dictated by the orientation of the two main proteins that make up the leaflet—collagen and elastin. For a healthy aortic leaflet, collagen is oriented in the circumferential direction and elastin is oriented in the radial direction. Although appearing similar in appearance in either direction, this anisotropy plays a vital role in valve biomechanics. As tested and observed on bench studies, applying loads on the leaflet in either one direction exhibits a different biomechanical behavior than the other direction. As blood continues to cyclically circulate and propel into the aortic root during systole, the ventricular side of the leaflets experience high shear forces or tangential forces that dictate the orientation of these proteins. Similarly, during diastole or leaflet closure, the aortic side experiences these forces.

When the leaflet material degrades, the environment created for the inflow of blood can be physiologically accurate to avoid any adverse flow effects or complications thereof. Hence, incorporating anisotropy in the design stage ensures that the gene processes that regulate neointima (native tissue) formation on these biodegradable materials, are guided with respect to fiber orientation. The neointima formation thus will be anisotropic as well, making the environment physiologically stable. Although the process incorporates this behavior within the design aspect, the feasibility of such a technique would need to be further supplemented and validated with animal studies.

Patient-specific design can be used for some embodiments, while others use standardized designs. Patient-specific anatomical dimensions can be measured using pre-procedural imaging and then designed for. Methods for such measurement include but are not limited to computerized tomography (CT) and magnetic resonance imaging (MRI). A personalized design could help ease the surgical procedure, decrease risk of complications, and negate the need for preparatory or follow-up procedures. For example, patient-specific design may not only ensure a perfect fit, but also may allow for partial heart valve replacements, with only the region that needs repair being affected.

Embodiments of this patient-specific valve design method involve a computational workflow/pipeline that generates valve designs to seamlessly fit and conform to the patient's anatomy. This is particularly important in complex and heterogeneous anatomies such as congenital heart defects. The design can further be tailored to other characteristics, which may include but are not limited to flow performance, washout characteristics to minimize the likelihood of thrombosis, and spatial degradation rates of the frame and the leaflets with variable material densities and thickness as a function of space.

Optimal design for long term performance of degradable valves relies on multiphysics simulation to predict how design coupled with long term material degradation and tissue growth will affect long term valve performance.

The multiphysics simulation described herein can incorporate long-term changes in mechanical properties due material degradation, growth of blood vessels, and tissue ingrowth/ongrowth. In term, these changes affect flow performance. The multiphysics simulation can include experimental measures of in vivo mechanical property changes (both elastic and post-yield) to improve the multiphysics simulation efficacy based on determined effects on leaflet/stent performance. For example, this can done using multi-generational material properties. Tissue growth and organ growth can be implemented in simulations using mixture theory. Together, these simulations can be used to predict changes in leaflet and stent mechanical properties over time. The multiphysics simulation can also be configured to account for changes in flow patterns through the valve due to changes in leaflet stiffness due to material degradation and tissue ongrowth. Furthermore, multiphysics simulation can also be configured to account for changes in materials properties due to degradation to determine durability.

One embodiment of the methods to design and manufacture a patient-specific device is as follows. First, the patient images, for example from CT or MRI, are collected and processed for use. Second, a computer model is generated. This is done based on the geometric and flow characteristics collected via patient image processing, and an appropriate template is chosen. This template valve will be parameterized to n number of variables of interest, depending on the given patient's case. An algorithm is then used to achieve the parameter variables to an optimal set to generate the optimal version of the valve based on said chosen template. This is done by iteratively running through the parameter space and assessing an objective function, which for example may minimize the likelihood of thrombosis, reduce the pressure gradient over the valve, or optimize the degradation profiles for new tissue growth. An optimized 3D printable file of the valve is then output and is subsequently printed, processed, and implanted into the patient for which it was designed.

Another embodiment of the methods to design and manufacture a patient-specific device is as follows. One embodiment of a patient-specific design method is as follows. In this embodiment, the 3D printed heart valves are designed to fit patient-specific anatomy, specifically the annulus to which the valve is attached. CT scans with contrast or MRI images from the patient are used as the base input data. These scans in DICOM format are then input to image design/segmentation software which could include, but is not limited to, Mimics (Materialise™), ScanIP (Synopsis™), 3D Slice, ITK-Snap, and any combination thereof. A variety of image processing algorithms including density-based thresholding, region growing and slice by slice editing are used to segment out the annulus and ventricle/atrial geometry as applicable. Best first elliptical annulus measurements are taken from which the geometry of the annulus will be created. This annulus will have a general elliptical cross-section that may change longitudinally. The final design, created using CAD for example in SolidWorks, or using image-based methods, is created and converted to STL format. This STL geometry is used to print the stent based from a bioresorbable material. Based on the vessel opening and the need for coaptation, the valve geometry. For the aortic, tricuspid, and pulmonic valve there will be three leaflets. The mitral valve will have two leaflets. The leaflets can have an open base that will allow them to be 3D printed from a different bioresorbable material than the stent. The leaflets can then be assembled onto the stent.

One particular family of embodiments comprises leaflets composed of APGD or MPGD, and a description of how this could be implemented is as follows. The development of APGD or MPGD for use as a material component of a heart valve device follows a multistep workflow, where PGD is first synthesized and then acrylated. The resultant resin can then be 3D printed and the printed device is postprocessed as needed. In brief, PGD can be synthesized using equimolar amounts of dodecanedioic acid and glycerol. These reagents are heated and stirred in an oil bath at 120° C. under constant flow of nitrogen for 24 hr. After 24 hr, nitrogen flow is removed, and the reaction is placed under −28 in Hg vacuum conditions for 36 hr. Acryloyle chloride is then added in a 0.18 mol/mol ratio to the hydroxyl groups on the PGD pre-polymer. 0.5 wt % Phenylbis(2,4,6-trimethylbenzoyl)phosphine oxide (BAPO) is added to the resin as a photoinitiator to allow for curing under 405 nm wavelength UV light exposure. 405 nm UV light is the typical wavelength of light used by commercial digital light projector (DLP) 3D printers such as 3D printer 700, including the Lumen X Bioprinter (Cellink) which has successfully been used in the past to print APGD. 0.5 vol % tartrazine is added to the resin prior to 3D printing as a photoabsorbent. This resin can then be used to print heart valve devices or components thereof on the Lumen X bioprinter. Printed devices or parts are then post processed via thermal curing, where prints are placed in an oven at 120° C. for up to 24 hr. While this method is utilized to manufacture some embodiments, others may utilize other materials including but not limited to resins with different acrylate crosslinkers to modulate material behavior and resins with different crosslinker chemistries such as thiolene interactions. Additional chemistries could utilize different photoinitiators for improved function or different UV wavelengths including but not limited to 2,2-Dimethoxy-2-phenylacetophenone (DMPA), monoacylphosphine oxide (MAPO), 2,2′-azobis[2-methyl-N-(2-hydroxyethyl) promionamide](VA-086), Riboflavin, and more.

A family of embodiments exists which utilize PCL for one or more components. Based upon the mechanical properties of PLC, it may serve especially well as a material used a frame subassembly. One potential 3D printing method, SLS, can be used to manufacture the PCL components utilized in some such embodiments within this family. For this printing method, the PCL is synthesized then milled into a powder using any number of methods which may include but are not limited to hammer milling, jet milling, and other processes which lend themselves to polymers like PCL, particularly those processes which can be conducted at cryogenic temperatures. The size of PCL powder is a factor which may likely affect printing, and parameters not just of size range but also average and standard deviation are data which will likely be considered and customized. The PCL powder may be mixed with one or more other materials prior to printing, for example to improve the behavior of the powder during printing or of the printed component. Other factors will likely be considered, such as the need to control the presence of water in the powder and in the air during printing, which may for example require desiccating the powder prior to printing and humidity control in the 3D printing environment. Pre-processing may not be required, and if it is used, it can include any number of steps for any number of purposes, including but not limited to mixing with additives and desiccating as described herein. The SLS printing process has numerous parameters than may potentially be control to ensure optimal printing, which may include but are not limited to processing chamber temperature and humidity, removal chamber temperature, laser intensity, speed, and wavelength, and layer thickness. After printing, there may be post-processing steps, such as sonication in an ethanol bath. Additional processes which may be conducted on the component include sterilization, which could utilize one or more of any number of methods, including but not limited to EtO, EtOH, UV, and NOx. Packaging is another potential consideration, as is quality control. Quality control for such components may include any number of methods to evaluate any number of parameters. For such SLS manufactured PCL components, this may include uCT to evaluate porosity and compression testing to evaluate mechanical strength.

One protocol which could be used to manufacture such an embodiment is as follows. The PCL is synthesized and subsequently milled (by third parties) to a powder in the range of 50 to 70 um using liquid jet milling at cryogenic temperatures. The PCL powder is then mixed with 4% hydroxylapatite (HA) by weight. The relative humidity (RH) of the powder mixture is then measured and reduced, if necessary, by storing it in a chamber sealed from the surrounding environment with consistent nitrogen flow. The RH must be below 15% and ideally below 10% prior to printing with it. A Formiga P110 (EOS) is used, which is installed in a cleanroom along with a continual powder supply module for the event power is lost temporarily. Before printing, the room, printer, and associated equipment is thoroughly cleaned using 70% ethanol. The dry powder mixture is then used to print one or more components. During the printing process, the printer's processing chamber is held at roughly 54 C, while the removal chamber is held at roughly 43 C.

The present disclosure provides many advantages over conventional devices which involve permanent devices. Embodiments of the present disclosure, however, will eventually be absorbed by the body and in its place, new tissue growth will serve the function the device once did. This lends itself particularly to pediatrics, where the patients are growing at rapid rates and permanent devices quickly become too small, increasing risk of adverse events and necessitating their replacement. The present disclosure provides a reliable treatment for the short term, while also bringing about the growth of new tissues to eventually create a self-sustaining treatment for the long term. This can eliminate the need for surgical reintervention, reducing risk of complications and overall treatment costs.

The unique use of 3D printing to manufacture valves composed of resorbable materials creates numerous advantages over existing methods. One such benefit is that 3D printing allows for the efficient manufacturing of patient-specific geometries. Patient-specific designs are numerous advantages, including more flexibility in what conditions and which patients can be treated and decreased risk of complications due to poor fit. Standardized geometries lend themselves to mass manufacturing and thus lower cost, as well as possibly a reduced regulatory burden, and some embodiments of the present invention conform to this business model. Another benefit of 3D printing is that it allows for a wider range of geometries than other current manufacturing methods such as via mold or electrospinning. It also allows for easier prototyping and less time between designing and manufacturing steps. For example, one embodiment may comprise an aortic valve that have dimensions that best replace the native valve depending on the patient's aortic roots as measured using CT. This valve being resorbable allows for regrowth and remodeling of the heart valve from the patients' cells and tissues, which, in this embodiment, accommodates somatic growth to ensure a functional valve for a growing pediatric patient.

Another benefit of the present disclosure over existing technologies is its ability to be customized and tuned, not just regarding geometry, but also regarding mechanical properties, thermodynamic properties, chemical properties, surface chemistry and morphology, and more to optimize the tissue interface and improve performance. The tunability of bulk and surface properties can be utilized to match the properties of surrounding tissues. Materials may be modified, for example through changing composition or processing, to optimize their properties, and hence behavior. Bioresorbable devices often perform best when their mechanical properties match those of the anatomy being mimicked or interfaced with, and modifications to parameters such as polymer chain length or crosslink density may aid in reducing the mismatch in behavior between device and tissue, for example elastomeric and viscoelastic behavior. Scaffolding applications have been shown to benefit from performing the functions of both structural support and communication with cells as would be supplied naturally by the extracellular matrix (ECM). One example of this is the possibility to encourage growth of specific cells, for example somatic growth which is important for pediatric patients, by functionalizing a polymer surface with selected peptides. Device designs can be altered to promote hemodynamics and durability. Different embodiments can therefore be designed to be ideal for different given applications, considering patient anatomy, age, specific condition/disease, as well as speed and cost factors, with the goal of choosing the optimal geometry, bulk and surface properties, and delivery mechanism for the application.

The present disclosure also provides commercial utility. Heart valve diseases and replacements are common, and the present invention aims to target this market, providing an ideal solution for a uniquely wide range of heart valve applications. Embodiments of the present disclosure can be used to treat any number of diseases which include but are not limited to regurgitation, valve insufficiency, bicuspid valve disease, valve stenosis, congenital heart diseases involving valve atresia, and valvular pathologies due to rheumatic fever. Embodiments of the present disclosure can be used to repair or replace any number of anatomies which include but are not limited to atrioventricular and semilunar valves, aortic, pulmonary, mitral, and tricuspid valves. Thus, different embodiments may have different numbers of leaflets. Designs with two and three leaflets are likely to be in higher demand than others.

Embodiments of the present disclosure also have commercial applications for patients and conditions that no existing devices are equipped to treat optimally, for example pediatric patients and those with uncommon diseases and/or anatomical geometries. Variations in deployment method between embodiments can further allow ease of handling for surgeons, interventional cardiologists, clinicians, and other medical professionals. Commercial applications can include both transcatheter heart valve replacement and percutaneous heart valve replacement, such as those performed transfemorally and transapically.

It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways. Also, it is to be understood that the phraseology and terminology employed herein are for the purposes of description and should not be regarded as limiting the claims.

Accordingly, those skilled in the art will appreciate that the conception upon which the application and claims are based may be readily utilized as a basis for the design of other structures, methods, and systems for carrying out the several purposes of the embodiments and claims presented in this application. It is important, therefore, that the claims be regarded as including such equivalent constructions.

Furthermore, the purpose of the foregoing Abstract is to enable the United States Patent and Trademark Office and the public generally, and especially including the practitioners in the art who are not familiar with patent and legal terms or phraseology, to determine quickly from a cursory inspection the nature and essence of the technical disclosure of the application. The Abstract is neither intended to define the claims of the application, nor is it intended to be limiting to the scope of the claims in any way.

1. A device comprising:
a flexible bioresorbable semilunar valve comprising:
a flexible circumferential body having a ring;
points extending from the ring along a longitudinal axis of the flexible bioresorbable semilunar valve; and
leaflets extending between the points.

2. The device of claim 1 further comprising a flexible cage disposed concentrically around the ring, wherein the flexible cage is configured to secure the flexible bioresorbable semilunar valve within a cardiac lumen; and
wherein the flexible cage is configured to transition from a collapsed delivery configuration to an expanded configuration and lock in the expanded configuration; and
wherein the flexible cage at least one of:
(i) comprises a patterned surface configured to provide friction with the cardiac lumen and to allow cellular ingrowth, wherein the patterned surface is functionalized with a pharmaceutical agent, and wherein the patterned surface comprises layers configured to be bioabsorbed over a period of time resulting in a release of the pharmaceutical agent;
(ii) comprises an annular member affixed to the flexible cage, wherein the annular member comprises a first end and an open second end, and wherein the first end of the annular member is configured to slide past the second end transitioning the flexible cage from the collapsed delivery configuration to the expanded configuration; or
(iii) comprises articulating joints configured to transition the flexible cage between the configurations, wherein the flexible cage further comprises struts that are substantially parallel in the collapsed delivery configuration, and are arranged in a diamond pattern in the expanded configuration.

3. The device of claim 1, wherein:
the leaflets are coupled to the points via peg-and-hole connection;
the leaflets are fused to the points via melting; or
the leaflets and the points are fused together via a gradient material transition from the leaflets to the points.

4. The device of claim 2, wherein the flexible cage of at least one of (ii) or (iii) further comprises a patterned surface functionalized with one or more of: proteins, peptides, cells, a pharmaceutical agent, biofactors, genetic materials, hydrogels, and biomaterials.

5. (canceled)

6. The device of claim 4, wherein the patterned surface is configured to promote one or more of: cellular ingrowth, tissue adhesion, and vascularization.

7. (canceled)

8. The device of claim 2, wherein the annular member of the flexible cage of (ii) further comprises interlocking teeth configured to permit the annular ring to transition only from the collapsed delivery configuration to the expanded configuration.

9. (canceled)

10. A system comprising:
the device of claim 2; and
a catheter configured to deliver the flexible bioresorbable semilunar valve and the flexible cage.

11. The system of claim 10, wherein the flexible cage comprises a shape memory material configured to expand at body temperature from the collapsed delivery configuration to the expanded configuration.

12. The system of claim 10 further comprising a balloon disposed in the flexible cage and configured to, upon inflating with a fluid, forcibly outwardly expand the flexible cage from the collapsed delivery configuration to the expanded configuration.

13. The device of claim 2 or claim 3, wherein a tongue is formed where each leaflet adjoins an adjacent leaflet; and
wherein each of the points of the flexible circumferential body comprises a groove configured to receive the tongue.

14. The device of claim 13 further comprising an elastic sleeve configured to be affixed circumferentially around the leaflets and the flexible circumferential body.

15. The device of claim 13 further comprising a reinforcement material;
wherein the flexible circumferential body comprises a first material;
wherein the leaflets comprises a second material;
wherein the first material is stiffer than the second; and
wherein the reinforcement material connects the first material and the second material.

16.-17. (canceled)

18. The device of claim 15, wherein the first material comprises polycaprolactone (PCL); and
wherein the second material comprises at least one of acrylated poly(glycerol dodecanedioate) (APGD) or methacrylated PGD (MPGD).

19. (canceled)

20. The device of claim 2, wherein the leaflets are coupled to the points with an adhesive, with an attachment component, or via a suture.

21.-25. (canceled)

26. The device of claim 2 or claim 3, wherein at least one of:
the leaflets are interconnected by a sleeve configured to be affixed over the flexible circumferential body;
the flexible circumferential body further comprises a patient-specific outer profile formed via additive manufacturing of a three-dimensional (3D) valve design determined based on a patient-specific geometry of a cardiac lumen; or
the leaflets are 3D printed by a process selected from the group consisting of extrusion, selective laser sintering (SLS), direct metal laser sintering (DMLS), digital light processing (DLP), fused deposition modeling (FDM), fused filament fabrication (FFF), PolyJet, stereolithography (SLA), multi jet fusion (MJF), electron beam melting (EBM), and solid freeform fabrication (SFF).

27.-31. (canceled)

32. The device of claim 3 further comprising a pharmaceutical agent embedded in the device.

33. The device of claim 32, wherein the device is configured to gradually release the pharmaceutical agent.

34. A method of manufacturing a device for use in cardiovascular interventions, the method comprising:
receiving a three-dimensional valve design; and
additively manufacturing a valve according to the three-dimensional valve design.

35.-40. (canceled)

41. A device for use in cardiovascular interventions comprising:
one or more valve leaflets comprising one or more bioresorbable materials; and
one or more biofactors selected from the group consisting of cells, growth factors, genetic material, pharmaceuticals, and a combination thereof,
wherein one or more growth factors are selected from the group consisting of peptides, proteins, amino acids, synthetic molecules, and a combination thereof.

42. The device of claim 41, wherein at least one of:
one or more of the valve leaflets are 3D printed;
the device is configured to be delivered transcatheter;
the device has shape memory properties;
one or more of the bioresorbable materials comprise a biologically resorbable polymer
one or more of the bioresorbable materials comprise a biologically resorbable metal;
the device further comprises a leaflet subassembly and a frame configured to support the leaflet subassembly;
the device further comprises a pharmaceutical active ingredient embedded in or attached to the device;
the device is tailored to a specific patient's anatomy; or
the device further comprises at least one of patterned surfaces or porous scaffolding.

43.-95. (canceled)
